(19) United States
(12) Patent Application Publication
(10) Pub. No.: US 2025/0188447 A1
(43) Pub. Date: Jun. 12, 2025

(54) MODULAR VECTOR (MODVEC) SYSTEM: A PLATFORM FOR CONSTRUCTION OF NEXT GENERATION EXPRESSION VECTORS

(71) Applicant: AMGEN INC., Thousand Oaks, CA (US)

(72) Inventors: Fuyi CHEN, Camarillo, CA (US); Marissa MOCK, Newbury Park, CA (US); Edward J. BELOUSKI, Thousand Oaks, CA (US)

(73) Assignee: AMGEN INC., Thousand Oaks, CA (US)

(21) Appl. No.: 18/844,915

(22) PCT Filed: Mar. 7, 2023
(86) PCT No.: PCT/US2023/063848

§ 371 Date: Sep. 6, 2024

Related U.S. Application Data
(60) Provisional application No. 63/317,949, filed on Mar. 8, 2022.

(51) Int. Cl. C12N 15/10; C07K 16/00; C12N 15/85

(52) U.S. Cl. CPC C12N 15/1082; C12N 15/1072; C12N 15/85; C07K 16/00; C12N 2800/107; C12N 2800/90; C12N 2830/15; C12N 2830/50; C12N 2840/203

(57) ABSTRACT

The present invention is directed to a method for optimizing the expression levels of a multi-chain protein wherein the protein comprises at least two different polypeptide chains. The generation of multi-chain proteins presents great challenges due to the pairing/folding of new quaternary structures composed of multiple polypeptide chains, particularly when pairing antibody heavy and light chains in a multispecific format. Of critical import to efficient and proper assembly of multiple polypeptide chain molecules is expression of the different chains at the proper ratio within the cell. The present invention addresses these issues.

### CROSS REFERENCE TO RELATED APPLICATION

The present application claims the benefit of priority to U.S. Provisional Patent Application No. 63/317,949, filed Mar. 8, 2022, the entirety of which is hereby incorporated by reference herein.

### INCORPORATION BY REFERENCE

Incorporated by reference in its entirety is a sequence listing in XML format, identifiable by the following file properties: Filename: A-2671-WO01-SEC_Sequence_Listing.XML; File size: 8,686 bytes; Created: Mar. 6, 2023.

### FIELD OF THE INVENTION

The present invention relates to a method for the preparation of standardized expression cassettes. The invention also relates to a method for recombining such standardized expression cassettes in vivo in a host cell.

### BACKGROUND TO THE INVENTION

Multispecific antibodies and antibody-like constructs possess several characteristics that are attractive to those developing therapeutic molecules. The clinical potential of multispecific antibodies that target multiple targets simultaneously like bispecific and trispecific antibodies shows great promise for targeting complex diseases. However, the generation of those molecules presents great challenges due to the pairing/folding of new quaternary structures composed of multiple polypeptide chains upon transfection into a single cell, particularly when pairing antibody heavy and light chains. Of critical import to efficient and proper assembly of multiple polypeptide chain molecules is expression of the different chains at the proper ratio within the cell.

The present invention addresses this issue using a modular vector (ModVec) system to rapidly build diverse expression vectors and vector libraries for recombinant protein production. ModVec enables rapid and high throughput construction of both simple and complex vector designs, enabling optimization of expression vectors for individual molecules, for specific large-molecule modalities, and/or for different expression hosts.

One major challenge in producing multichain molecules is varied product quality caused by imbalanced chain expression ratio, and this ratio is molecule specific as well as specific to the cell-host development process including the expression vector elements. Currently there is no approach to determine the optimal molecule-specific chain expression ratio with high throughput. ModVec vector library provide a platform to rapidly determine the best expression configuration for individual multichain molecule. ModVec can be used in more generalized applications to assemble complex large DNA molecules for purposes other than expression vectors for antibody therapeutics.

### SUMMARY OF THE INVENTION

In one aspect, the present invention is directed to a method for expressing a multi-chain protein comprising at least two different polypeptide chains comprising
a. providing polynucleotide sequences that encode for the at least two different polypeptides, wherein said polynucleotide sequences are flanked on both 5′ and 3′ sides by a type IIS restriction endonuclease cleavage site followed by a recognition site thereof;
b. providing two or more sets of element sequences; wherein each set of element sequences together comprise at least one functional expression cassette,
wherein each element sequence is flanked on both 5′ and 3′ sides by a type IIS restriction endonuclease cleavage site followed by a recognition site thereof,
wherein the type IIS restriction endonuclease recognition sites and cleavage sites are selected so that the sets of element sequences may be assembled into a functional expression cassette;
c. providing at least one backbone entry vector, wherein the backbone entry vector comprises a plasmid comprising at least one type IIS restriction endonuclease cleavage site followed by a recognition site thereof and sequences for maintenance of the vector in bacterial cells;
d. assembling the polynucleotide sequences that encode for the at least two different polypeptides with the two or more sets of element sequences to generate functional expression cassettes in the backbone entry vector, using a method based on the use of type IIS restriction enzyme digestion and ligation via the cleavage sites and overhangs resulting in ligated vectors comprising at least two functional expression cassettes that are capable of expressing the at least two different polypeptide chains;
e. transfecting mammalian cells with the ligated vectors;
f. comparing the levels of preferred product produced and levels of impurities of each of the at least two polypeptides; and
g. identifying the optimal expression ratio of the two polypeptides for the transfected mammalian cells and identifying the transfected mammalian cells that have the optimal expression ratio of the at least two polypeptide chains.

In one embodiment, the method further comprises:
h. identifying the arrangement of vector elements that provides the optimal expression ratio of the at least two polypeptide chains.

In one aspect, the present invention is directed to a vector comprising the arrangement of vector elements that provides the optimal expression ratio of the at least two polypeptide chains.

In one embodiment, the element sequences comprise at least two promoter sequences and at least two polyA sequences.

In one embodiment, the levels of preferred product produced are measured by a method selected from the group consisting of cation exchange chromatography (reduced or non-reduced), mass spectrometry, any other chromatographic separation, or a combination thereof.

In one embodiment, none of the type IIS restriction endonuclease cleavage sites produce 5′ four nucleotide overhangs selected from the group consisting of GTAA, TCCA, and CACA upon cleavage by the type IIS restriction endonuclease.

In one embodiment, the type IIS restriction endonuclease cleavage site is selected from the group consisting of:
AGGT, AGTA, ATCA, CAGT, CCAT, GAAT, GAGG, GGCA, GGTC, TAGC, TCTT, GGAG, and CCAC.

In one embodiment, the mammalian cells are selected from the group consisting of CHO cells, CHOK1 cells, DXB-11, DG-44, COS-7, HEK293, BHK, TM4, CV1, VERO-76, HELA, MDCK, BRL 3A, W138, Hep G2, MMT 060562, TRI cells, MRC 5 cells, FS4 cells, and mammalian myeloma cells.

In one embodiment, the optimal expression ratio of the at least two polypeptides is selected from the group consisting of 1:1, 1:2, and 1:3.

In one embodiment, the multi-chain protein comprises a first antibody heavy chain, a first antibody light chain, a second antibody heavy chain, and a second antibody light chain, wherein the first antibody heavy chain associates with the first antibody light chain to bind a first antigen or epitope and the second antibody heavy chain associates with the second antibody light chain to bind a second antigen or epitope, wherein the optimal expression ratio of the first antibody heavy chain, the first antibody light chain, the second antibody heavy chain, and the second antibody light chain is 1:1:1:1.

In one embodiment, the multi-chain protein comprises a first antibody heavy chain, a second antibody heavy chain, and a common antibody light chain, wherein the first antibody heavy chain associates with the common antibody light chain to bind a first antigen or epitope and the second antibody heavy chain associates with the common antibody light chain to bind a second antigen or epitope, wherein the optimal expression ratio of the first antibody heavy chain, the second antibody heavy chain, and the common antibody light chain is 1:1:2.

In one embodiment, the multi-chain protein comprises an antibody heavy chain, a first antibody light chain, a modified antibody heavy chain, and a second antibody light chain,
wherein the modified antibody heavy chain comprises, N-terminal to C-terminal, one of the following structures selected from the following group:
VH-CH1-binding domain (BD)-hinge-CH2-CH3;
BD-VH-CH1-hinge-CH2-CH3; and
VH-CH1-hinge-CH2-CH3-BD;
wherein the BD is selected either a single-chain Fv (scFv) or a single-chain Fab (scFab);
wherein the antibody heavy chain associates with the first antibody light chain to bind a first antigen or epitope and the VH of the modified antibody heavy chain associates with the second antibody light chain to bind a second antigen or epitope, wherein the BD binds to a third antigen or epitope, and
wherein the optimal expression ratio of the first antibody heavy chain, the first light chain, the modified antibody heavy chain, and the second antibody light chain is 1:1:1:1.

In one embodiment, the multi-chain protein comprises an antibody heavy chain, a modified antibody heavy chain, and a common antibody light chain,
wherein the modified antibody heavy chain comprises, N-terminal to C-terminal, one of the following structures selected from the following group:
VH-CH1-binding domain (BD)-hinge-CH2-CH3;
BD-VH-CH1-hinge-CH2-CH3; and
VH-CH1-hinge-CH2-CH3-BD;
wherein the BD is selected either a single-chain Fv (scFv) or a single-chain Fab (scFab);
wherein the antibody heavy chain associates with the common antibody light chain to bind a first antigen or epitope and the VH of the modified antibody heavy chain associates with the common antibody light chain to bind the first antigen or epitope, wherein the BD binds to a second antigen or epitope, and
wherein the optimal expression ratio of the first antibody heavy chain, the modified antibody heavy chain, and the common antibody light chain is 1:1:2.

### BRIEF DESCRIPTION OF THE DRAWINGS

FIGS. 1A-1E depicts the Modular vector platform for high throughput vector engineering (“ModVec”). FIG. 1A) Example sequence of DNA module. Each module (depicted in Ns and grey shade) is flanked by Golden Gate adaptors (boxed region) which contain recognition site for BsmBI (highlighted in bold). BsmBI cut sites are marked with \hat{ } and overhangs generated through BsmI digestion are underlined. FIG. 1B) Assembly of an expression vector is achieved through Golden Gate reaction using BsmBI with vector elements flanked by predefined complementary overhangs plus an expression vector backbone. FIG. 1C) Schematic of an 11 kb 11-piece (including vector backbone) ModVec assembly. ITR: inverted terminal repeats of piggyBac transposon. CDS: coding sequence. FIG. 1D) All 19 constructs were successfully assembled using ModVec through one round of cloning. 96% of colonies (109 out of 114) had correctly assembled constructs. FIG. 1E) Schematic of the one-tube vector library assembly. FIG. 1F) Efficient one-tube vector library construction using ModVec; 69 out of 72 possible constructs were obtained by evaluating 350 colonies.

FIGS. 2A-2D depicts that Choice of promoter has significant impact on manufacturability. FIG. 2A) Schematic of vector configurations. Both LC and HC genes were under the control of the same promoter, either Promoter 1 or Promoter 2. Impact of promoter on the manufacturability of standard mAb A, bsAb B and bsAb C was shown in FIG. 2B, FIG. 2C, and FIG. 2D respectively.

FIGS. 3A-3H depicts Improving productivity of a four-chain Hetero-IgG using vector configuration library screening. FIG. 3A) Schematics of vector configurations for three scale up pools. FIG. 3B) LC1: LC2 ratio of ProA-purified samples from pools 905, 910, and 911 before scaling up for further analysis. FIG. 3C) ProA yield of scaled up pools 905, 910, and 911. FIG. 3D) Cation exchange purification recovery rate of the three pools. FIG. 3E) Final yield of correctly assembled Hetero-IgG-D after cation exchange. Cation exchange chromatograms for ProA-purified samples from pools 905, 910, and 911 are shown in FIG. 3F, FIG. 3G, and FIG. 3H, respectively. The identity of each peak was determined by mass spectrometry.

FIGS. 4A-4H depicts Improving productivity of an asymmetric trispecific molecule using vector configuration library screening. FIG. 4A) Schematics of vector configuration library design. Three different groups of configurations, single LC, double LC A, and double LC B were tested. In each configuration, three different promoters, Promoter 1, Promoter 2, and Promoter 3 were used to control the transcription of the polypeptide chains. FIG. 4B) ProA yield of all vector configurations. Average ProA yield of vector configurations in double LC A, double LC B, and single LC groups was 117.2±43.4 mg/L, 112.6±39.2 mg/L, and 40.7±20.73 mg/L, respectively. Values expressed as mean±standard deviation. FIG. 4C) Product quality of ProA-purified samples from all vector configurations. Average nrMCE % MP of vector configurations in double LC A, double LC B, single LC was 75.6±14.9%, 72.4±16.6%, and 71.7±21.6%, respectively. FIG. 4D) Final yield of correctly assembled tsAb E of all vector configurations. Average final yield of vectors configurations in double LC A, double LC B, and single LC groups was 88.7±33.6 mg/L, 81.4±35.6 mg/L, and 28.5±16.8 mg/L, respectively. FIG. 4E) ProA yield of stable pools generated from default and optimal configurations using standard cell line development protocol. The ProA yield of the optimal pool had 124% increase in ProA yield compared to the default pool. FIG. 4F) Product quality of ProA samples from the default and the optimal pool was comparable. FIG. 4G) The final yield of tsAb E was 114 mg/L from the default pool and 257 mg/L from the optimal pool. The optimal pool had 125% increase in final yield compared to the default pool. FIG. 4H) Schematics of optimal vector configuration identified through library screening and our platform vector configuration.

FIGS. 5A-5D depicts the Vector configuration library screening for a difficult to express Hetero-IgG. FIG. 5A) Schematics of vector configurations used. Expression of the Hetero-IgG D involved co-transfection of two bicistronic vectors containing genes for all four polypeptide chains. Config 1 vectors have the LC and HC gene from the same mAb in the same bicistronic vector. Config 2 vectors have LC and HC genes in separate bicistronic vector. FIG. 5B) ProA yield of all recovered stable CHO cell pools; 29 out of 44 pools recovered. FIG. 5C) LC1: LC2 ratios of ProA-purified samples from all pools. Pools marked with asterisks were scaled up for further analysis. FIG. 5D) Product quality of all pools measured by non-reducing MCE main peak and SEC main peak.

FIGS. 6A-6G depicts that Gene position in the expression vector can have significant impact on manufacturability. FIG. 6A) Schematic of DA2 and DB2 configuration. Both configurations used Promoter 2 to drive the expression of all polypeptide chains. FIG. 6B) Pool DA2 had higher ProA yield than pool DB2. FIG. 6C) Product quality of ProA purified samples from pools DA2 and DB2 was comparable. FIG. 6D) Pool DA2 had higher final yield of tsAb E than pool DB2. FIG. 6E) Electropherograms (non-reducing microcapillary electrophoresis) of ProA-purified samples from pool DA2. The relative peak area for half mAb1 was 10.1 and half mAb2 was 7.4 for ProA-purified samples. FIG. 6F) Electropherograms (non-reducing microcapillary electrophoresis) of ProA-purified samples from pool DB2. The relative peak area for half mAbl was 5.8 and half mAb2 was 9.8. FIG. 6G) Half mAb1: half mAb2 relative peak ratio for ProA purified samples from pools DA2 was 1.4 while this value was 0.6 for ProA purified samples for pool DB2.

### DETAILED DESCRIPTION OF THE INVENTION

The method of the invention allows the production of expression cassettes of interest from sets of element sequences by assembling nucleic acid fragment constructs via single-stranded overhangs formed at both ends of the fragments using type II restriction endonucleases. In the invention, type II is restriction enzymes may be used. The type II restriction endonuclease recognition site is a recognition site of a restriction endonuclease recognizing a double-stranded DNA and cleaving the double-stranded DNA at a cleavage site that is outside the recognition site on the double stranded DNA. The type II restriction endonuclease cleaves such that, depending on the specific type II restriction endonuclease, overhangs of from 3 to 6 nucleotides are produced. Typically, in the method of the invention, enzymes giving rise to 4 nucleotide overhangs may be used. However, it is also possible to use type II endonucleases producing longer single-stranded overhangs. The nucleotide range that forms the overhangs upon cleavage is referred to herein as cleavage site. Since the nucleotides of the cleavage site are not part of the recognition site, they can be chosen as desired without destroying cleavage activity of the type II restriction endonuclease

For practicing the invention, any type II restriction enzyme that provides “sticky” ends sufficient for efficient ligation at its cleavage sites can be used. A selection of such enzymes is provided on the REBASE webpage (rebase.neb.com/cgi-bin/asymmlist) and in the review of Szybalsky et al. (1991,Gene, 100:13-26). Most preferred are the following type II restriction endonucleases: Bsal, Bbsl, BsmBI, Sapl, BspMI, Aarl, Esp31, Bpil, and Hgal. Many of the cited restriction endonucleases are available from New England Biolabs. Sources of these enzymes can also be found on the REBASE webpage mentioned above. Type II restriction enzymes with asymmetric recognition sites (e.g. those shown in this webpage) that have cleavage site outside of recognition site and provide upon cleavage of at least three, preferably 4 or more nucleotide residues overhangs (e.g. BsmBI, BN736I; BpuAI, VpaK321, SfaNI, etc.) can be used in the invention. In a one embodiment, the Type II restriction endonuclease is BsmBI.

It is recommended that the recognition site contains at least 4, more preferably at least 6 or more base pairs in order to minimize the chance for such site to be found in a sequence portion of interest. Type II restriction nucleases with 5 bp recognition sites (e.g. SfaNI) also can be used. Type II restriction endonucleases that produce 4 nt single-stranded overhangs at the extremities of digested fragments can theoretically generate ends with 256 possible sequences. Type II restriction enzymes having even longer recognition sites, e.g. comprising ten or more base pairs have been engineered. In one embodiment, the recognition site is 5′-CGTCTC-3′. In one embodiment, upon cleavage by the IIS restriction endonuclease, the 5′ overhang is four nucleotides in length but is not selected from the group consisting of GTAA, TCCA, and CACA. In one embodiment, upon cleavage by the IIS restriction endonuclease, the 5′ overhang is four nucleotides in length and is selected from the group consisting of AGGT, AGTA, ATCA, CAGT, CCAT, GAAT, GAGG, GGCA, GGTC, TAGC, TCTT, GGAG, and CCAC.

Examples of ligases to be used in the invention include T4 DNA ligase, E.coli DNA ligase, Taq DNA ligase, all of which are commercially available from New England Biolabs.

In one aspect, the present invention is directed to a method for expressing a multi-chain protein comprising at least two different polypeptide chains comprising
a. providing polynucleotide sequences that encode for the at least two different polypeptides, wherein said polynucleotide sequences are flanked on both 5′ and 3′ sides by a type IIS restriction endonuclease cleavage site followed by a recognition site thereof;
b. providing two or more sets of element sequences; wherein each set of element sequences together comprise at least one functional expression cassette,
wherein each element sequence is flanked on both 5′ and 3′ sides by a type IIS restriction endonuclease cleavage site followed by a recognition site thereof,
wherein the type IIS restriction endonuclease recognition sites and cleavage sites are selected so that the sets of element sequences may be assembled into a functional expression cassette;
c. providing at least one backbone entry vector, wherein the backbone entry vector comprises a plasmid comprising at least one type IIS restriction endonuclease cleavage site followed by a recognition site thereof and sequences for maintenance of the vector in bacterial cells;
d. assembling the polynucleotide sequences that encode for the at least two different polypeptides with the two or more sets of element sequences to generate functional expression cassettes in the backbone entry vector, using a method based on the use of type IIS restriction enzyme digestion and ligation via the cleavage sites and overhangs resulting in ligated vectors comprising at least two functional expression cassettes that are capable of expressing the at least two different polypeptide chains;
e. transfecting mammalian cells with the ligated vectors;
f. comparing the levels of preferred product produced and levels of impurities of each of the at least two polypeptides; and
g. identifying the optimal expression ratio of the two polypeptides for the transfected mammalian cells and identifying the transfected mammalian cells that have the optimal expression ratio of the at least two polypeptide chains.

In one embodiment, the method further comprises:
h. identifying the arrangement of vector elements that provides the optimal expression ratio of the at least two polypeptide chains.

In one aspect, the present invention is directed to a vector comprising the arrangement of vector elements that provides the optimal expression ratio of the at least two polypeptide chains.

Each set of element sequences will typically be capable of being assembled as an expression cassette. An expression cassette in the context of this invention is intended to indicate a nucleic acid sequence that directs a cell's machinery to make RNA and protein. Typically, an expression cassette will comprise a coding sequence and the sequences controlling expression of that coding sequence. Typically, an expression cassette may comprise at least a promoter, an open reading frame and a terminator sequence. Other element sequences include control sequence, insulator sequence, bar code DNA sequence, primer sequences, promoter sequences, polyA sequences, and IRES sequences, and a mammalian selectable marker sequence. In one embodiment, the element sequences comprise at least two promoter sequences and at least two poly A sequences.

The term “control sequences” is defined herein to include all components, which are necessary or advantageous for the production of mRNA or a polypeptide, either in vitro or in a host cell. Each control sequence may be native or foreign to the nucleic acid sequence encoding the polypeptide. Such control sequences include, but are not limited to, a leader, Shine-Delgarno sequence, optimal translation initiation sequences (as described in Kozak, 1991, J. Biol. Chem. 266:19867-19870), a polyadenylation sequence, a pro-peptide sequence, a pre-pro-peptide sequence, a promoter, a signal sequence, and a transcription termination signal. At a minimum, the control sequences typically include a promoter, and a transcriptional stop signal (terminator or termination signal). Translational start and stop signals may typically also be present. Control sequences may be optimized to their specific purpose.

The term “promoter” is defined herein as a DNA sequence that binds RNA polymerase and directs the polymerase to the correct downstream transcriptional start site of a nucleic acid sequence encoding a biological compound to initiate transcription. RNA polymerase effectively catalyzes the assembly of messenger RNA complementary to the appropriate DNA strand of a coding region. The term “promoter” will also be understood to include the 5′-non-coding region (between promoter and translation start) for translation after transcription into mRNA, cis-acting transcription control elements such as enhancers, and other nucleotide sequences capable of interacting with transcription factors.

The method of the invention is typically carried out such that the elements of an expression cassette are assembled in a backbone entry vector such that they are in operable linkage. The term “operable linkage” or “operably linked” or the like are defined herein as a configuration in which a control sequence is appropriately placed at a position relative to the coding sequence of the DNA sequence such that the control sequence directs the production of an mRNA or a polypeptide.

“Insulator sequence” or “insulators” are nucleic acid segments that reduce or eliminate transcription from adjacent regions from affecting the nucleic acid segment to which the insulator is associated. Insulators preferably are placed upstream of other control sequences and/or downstream of genes. Insulators are preferably placed between different genes, transcription units, or genetic domains to reduce or prevent interference of the adjacent expression sequences.

“Enhancer sequence” or “enhancers” function to increase the transcription from promoters in proximity to the enhancer. Enhancers can function both upstream and downstream from a gene, and in either orientation.

“Barcode DNA sequence” or “barcodes” can be used to identify nucleic acid molecules, for example, where sequencing can reveal a certain barcode coupled to a nucleic acid molecule of interest. In some instances, a sequence-specific event can be used to identify a nucleic acid molecule, where at least a portion of the barcode is recognized in the sequence-specific event, e.g., at least a portion of the barcode can participate in a ligation or extension reaction. The barcode can therefore allow identification, selection or amplification of DNA molecules that are coupled thereto.

“IRES” or “internal ribosome entry site” means a site that allows internal ribosome entry sufficient to initiate translation in an assay for cap-independent translation, such as the bicistronic reporter assay described herein. The presence of an IRES allows cap-independent translation of a linked protein-encoding sequence that otherwise would not be translated.

A “selectable marker gene” or “selectable marker” encodes a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells; (b) complement auxotrophic deficiencies of the cell; or (c) supply critical nutrients not available from complex or defined media. Specific selectable markers are the kanamycin resistance gene, the ampicillin resistance gene, and the tetracycline resistance gene. Advantageously, a neomycin resistance gene may also be used for selection in both prokaryotic and eukaryotic host cells.

Other selectable genes may be used to amplify the gene that will be expressed. Amplification is the process wherein genes that are required for production of a protein critical for growth or cell survival are reiterated in tandem within the chromosomes of successive generations of recombinant cells. Examples of suitable selectable markers for mammalian cells include dihydrofolate reductase (DHFR) and promoterless thymidine kinase genes. Mammalian cell transformants are placed under selection pressure wherein only the transformants are uniquely adapted to survive by virtue of the selectable gene present in the vector. Selection pressure is imposed by culturing the transformed cells under conditions in which the concentration of selection agent in the medium is successively increased, thereby leading to the amplification of both the selectable gene and the DNA that encodes another gene, such as one or more components of the multi-chain proteins constructs described herein. As a result, increased quantities of a polypeptide are synthesized from the amplified DNA.

Accordingly, an element in the context of this invention is any constituent of an expression cassette. A set of elements is a group of elements that together may give rise to an expression cassette. The method of the invention requires that provision of two sets of element sequences. This means that enough elements are to be provided so that at least two different expression cassettes may result. This implies that there must be at least two different species of at least one element provided. That is to say, one promoter, taken in combination with two ORFs and two termination signals constitutes two sets of element sequences for the purposes of this invention.

In a method of the invention, typically at least two of the sets of element sequences comprise a promoter element, an open reading frame element and a termination signal element.

In a method according to the invention, one or more of the sets of elements may comprise “partial” element sequences, such as UTRs, signal peptides and split-open reading frames.

Each set of element sequences is provided in a form so that the set may be assembled into a functional expression cassette in a backbone entry vector. Typically then, each element is flanked by on both sides by a type II restriction endonuclease cleavage site followed by the recognition site thereof, the type II restriction endonuclease recognition sites and cleavage sites being selected so that the sets of element sequences may be assembled into a functional expression cassette. Each element sequence and flanking sequence therefore typically comprises in order from one end to the other: type II restriction endonuclease recognition site; cleavage site thereof; element sequence; type II restriction endonuclease cleavage site; recognition site thereof.

Accordingly, the sets of elements are prepared or provided in a suitable vector with type II restriction endonuclease recognition sites and standardized cleavage sites (preferably 4-bp), selected such that after assembly, for example using a one-pot approach, such as Golden gate cloning, a functional expression cassette is formed.

A set of backbone entry vectors is prepared or provided. These vectors comprise contain left and right connector sequences suitable for assembly using sequence homology.

A subset of element sequences is selected together with backbone (bbn) entry vectors. These may be assembled, for example using Golden Gate cloning, resulting in functional expression cassettes comprised within the backbone entry vectors.

Exemplary host cells include prokaryote, yeast, or higher eukaryote cells. Prokaryotic host cells include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacillus, such as B. subtilis and B. licheniformis, Pseudomonas, and Streptomyces. Eukaryotic microbes such as filamentous fungi or yeast are suitable cloning or expression hosts for recombinant polypeptides. Saccharomyces cerevisiae, or common baker's yeast, is the most commonly used among lower eukaryotic host microorganisms. However, a number of other genera, species, and strains are commonly available and useful herein, such as Pichia, e.g. P. pastoris, Schizosaccharomyces pombe; Kluyveromyces, Yarrowia; Candida; Trichoderma reesia; Neurospora crassa; Schwanniomyces, such as Schwanniomyces occidentalis; and filamentous fungi, such as, e.g., Neurospora, Penicillium, Tolypocladium, and Aspergillus hosts such as A. nidulans and A. niger.

Host cells for the expression of glycosylated antigen binding proteins can be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection of such cells are publicly available, e.g., the L-1 variant of Autographa californica NPV and the Bm-5 strain of Bombyx mori NPV.

Vertebrate host cells are also suitable hosts, and recombinant production of antigen binding proteins from such cells has become routine procedure. Mammalian cell lines available as hosts for expression are well known in the art and include, but are not limited to, immortalized cell lines available from the American Type Culture Collection (ATCC), including but not limited to Chinese hamster ovary (CHO) cells, including CHOK1 cells (ATCC CCL61), DXB-11, DG-44, and Chinese hamster ovary cells/-DHFR (CHO, Urlaub et al., Proc. Natl. Acad. Sci. USA 77:4216, 1980); monkey kidney CV1 line transformed by SV40 (COS-7, ATCC CRL 1651); human embryonic kidney line (293 or 293 cells subcloned for growth in suspension culture, (Graham et al., J. Gen Virol. 36:59, 1977); baby hamster kidney cells (BHK, ATCC CCL 10); mouse sertoli cells (TM4, Mather, Biol. Reprod. 23:243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., Annals N.Y Acad. Sci. 383:44-68, 1982); MRC 5 cells or FS4 cells; mammalian myeloma cells, and a number of other cell lines. In certain embodiments, cell lines may be selected through determining which cell lines have high expression levels and constitutively produce multi-chain proteins of the present invention. In another embodiment, a cell line from the B cell lineage that does not make its own antibody but has a capacity to make and secrete a heterologous antibody can be selected. CHO cells are host cells in some embodiments for expressing the multi-chain proteins of the invention.

Host cells are transformed or transfected with the above-described nucleic acids or vectors for production of multi-chain proteins and are cultured in conventional nutrient media modified as appropriate for inducing promoters, selecting transformants, or amplifying the genes encoding the desired sequences. In addition, novel vectors and transfected cell lines with multiple copies of transcription units separated by a selective marker are particularly useful for the expression of antigen binding proteins. Thus, the present invention also provides a method for preparing a multi-chain proteins described herein comprising culturing a host cell comprising one or more expression vectors described herein in a culture medium under conditions permitting expression of the multi-chain proteins encoded by the one or more expression vectors; and recovering the multi-chain proteins from the culture medium.

The host cells used to produce the antigen binding proteins of the invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44, 1979; Barnes et al., Anal. Biochem. 102:255, 1980; U.S. Pat. Nos. 4,767,704; 4,657,866; 4,927,762; 4,560,655; or 5,122,469; WO90103430; WO 87/00195; or U.S. Patent Re. No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Upon culturing the host cells, the multi-chain proteins can be produced intracellularly, in the periplasmic space, or directly secreted into the medium. If the antigen binding protein is produced intracellularly, as a first step, the particulate debris, either host cells or lysed fragments, is removed, for example, by centrifugation or ultrafiltration. The bispecifc antigen binding protein can be purified using, for example, hydroxyapatite chromatography, cation or anion exchange chromatography, or affinity chromatography, using the antigen(s) of interest or protein A or protein G as an affinity ligand. Protein A can be used to purify proteins that include polypeptides that are based on human γ1, γ2, or γ4 heavy chains (Lindmark et al., J. Immunol. Meth. 62:1-13, 1983). Protein G is recommended for all mouse isotypes and for human γ3 (Guss et al., EMBO J. 5:15671575, 1986). The matrix to which the affinity ligand is attached is most often agarose, but other matrices are available. Mechanically stable matrices such as controlled pore glass or poly (styrenedivinyl) benzene allow for faster flow rates and shorter processing times than can be achieved with agarose. Where the protein comprises a CH3 domain, the Bakerbond ABX™ resin (J. T. Baker, Phillipsburg, N.J.) is useful for purification. Other techniques for protein purification such as ethanol precipitation, Reverse Phase HPLC, chromatofocusing, SDS-PAGE, and ammonium sulfate precipitation are also possible depending on the particular multi-chain proteins to be recovered.

As used herein, the term “antibody” refers to a tetrameric immunoglobulin protein comprising two light chain polypeptides (about 25 kDa each) and two heavy chain polypeptides (about 50-70 kDa each). The term “light chain” or “immunoglobulin light chain” refers to a polypeptide comprising, from amino terminus to carboxyl terminus, a single immunoglobulin light chain variable region (VL) and a single immunoglobulin light chain constant domain (CL). The immunoglobulin light chain constant domain (CL) can be kappa (κ) or lambda (λ). The term “heavy chain” or “immunoglobulin heavy chain” refers to a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu (μ), delta (Δ), gamma (γ), alpha (α), and epsilon (ε), and define the antibody's isotype as IgM, IgD, IgG, IgA, and IgE, respectively. The IgG-class and IgA-class antibodies are further divided into subclasses, namely, IgG1, IgG2, IgG3, and IgG4, and IgA1 and IgA2, respectively. The heavy chains in IgG, IgA, and IgD antibodies have three domains (CH1, CH2, and CH3), whereas the heavy chains in IgM and IgE antibodies have four domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CHI domain (i.e. between the light and heavy chain) and between the hinge regions of the antibody heavy chains.

In a human antibody, CHI means a region having the amino acid sequence at positions 118 to 215 of the EU index. A highly flexible amino acid region called a “hinge region” exists between CHI and CH2. CH2 represents a region having the amino acid sequence at positions 231 to 340 of the EU index, and CH3 represents a region having the amino acid sequence at positions 341 to 446 of the EU index.

“CL” represents a constant region of a light chain. In the case of a k chain of a human antibody, CL represents a region having the amino acid sequence at positions 108 to 214 of the EU index. In a 2 chain, CL represents a region having the amino acid sequence at positions 108 to 215.

Both the EU index as in Kabat et al., Sequences of Proteins of Immunological Interest, 5th Ed. Public Health Service, National Institutes of Health, Bethesda, MD (1991) and AHo numbering schemes (Honegger A. and Plückthun A. J Mol Biol. 2001 Jun. 8;309(3):657-70) can be used in the present invention. Amino acid positions and complementarity determining regions (CDRs) and framework regions (FR) of a given antibody may be identified using either system. For example, EU heavy chain positions of 39, 44, 183, 356, 357, 360, 370, 392, 399, and 409 are equivalent to AHo heavy chain positions 46, 51, 230, 484, 485, 491, 501, 528, 535, and 551, respectively.

In one embodiment, the optimal expression ratio of the at least two polypeptides is selected from the group consisting of 1:1, 1:2, and 1:3.

In one embodiment, the multi-chain protein comprises a first antibody heavy chain, a first antibody light chain, a second antibody heavy chain, and a second antibody light chain, wherein the first antibody heavy chain associates with the first antibody light chain to bind a first antigen or epitope and the second antibody heavy chain associates with the second antibody light chain to bind a second antigen or epitope, wherein the optimal expression ratio of the first antibody heavy chain, the first antibody light chain, the second antibody heavy chain, and the second antibody light chain is 1:1:1:1.

In one embodiment, the multi-chain protein comprises a first antibody heavy chain, a second antibody heavy chain, and a common antibody light chain, wherein the first antibody heavy chain associates with the common antibody light chain to bind a first antigen or epitope and the second antibody heavy chain associates with the common antibody light chain to bind a second antigen or epitope, wherein the optimal expression ratio of the first antibody heavy chain, the second antibody heavy chain, and the common antibody light chain is 1:1:2.

In one embodiment, the multi-chain protein comprises an antibody heavy chain, a first antibody light chain, a modified antibody heavy chain, and a second antibody light chain,
wherein the modified antibody heavy chain comprises, N-terminal to C-terminal, one of the following structures selected from the following group:
VH-CH1-binding domain (BD)-hinge-CH2-CH3;
BD-VH-CHI-hinge-CH2-CH3; and
VH-CH1-hinge-CH2-CH3-BD;
wherein the BD is selected either a single-chain Fv (scFv) or a single-chain Fab (scFab);
wherein the antibody heavy chain associates with the first antibody light chain to bind a first antigen or epitope and the VH of the modified antibody heavy chain associates with the second antibody light chain to bind a second antigen or epitope, wherein the BD binds to a third antigen or epitope, and
wherein the optimal expression ratio of the first antibody heavy chain, the first light chain, the modified antibody heavy chain, and the second antibody light chain is 1:1:1:1.

In one embodiment, the multi-chain protein comprises an antibody heavy chain, a modified antibody heavy chain, and a common antibody light chain,
wherein the modified antibody heavy chain comprises, N-terminal to C-terminal, one of the following structures selected from the following group:
VH-CH1-binding domain (BD)-hinge-CH2-CH3;
BD-VH-CHI-hinge-CH2-CH3; and
VH-CHI-hinge-CH2-CH3-BD;
wherein the BD is selected either a single-chain Fv (scFv) or a single-chain Fab (scFab);
wherein the antibody heavy chain associates with the common antibody light chain to bind a first antigen or epitope and the VH of the modified antibody heavy chain associates with the common antibody light chain to bind the first antigen or epitope, wherein the BD binds to a second antigen or epitope, and
wherein the optimal expression ratio of the first antibody heavy chain, the modified antibody heavy chain, and the common antibody light chain is 1:1:2.

A “binding domain” or “BD”, may typically comprise an antibody light chain variable region (VL) and an antibody heavy chain variable region (VH); however, it does not have to comprise both. Fd fragments, for example, have two VH regions and often retain some antigen-binding function of the intact antigen-binding domain. Additional examples for the format of antibody fragments, antibody variants or binding domains include (1) a Fab fragment, a monovalent fragment having the VL, VH, CL and CHI domains; (2) a F(ab′)2 fragment, a bivalent fragment having two Fab fragments linked by a disulfide bridge at the hinge region; (3) an Fd fragment having the two VH and CHI domains; (4) an Fv fragment having the VL and VH domains of a single arm of an antibody, (5) a dAb fragment (Ward et al., (1989) Nature 341:544-546), which has a VH domain; (6) an isolated complementarity determining region (CDR), and (7) a single chain Fv (scFv), the latter being preferred (for example, derived from an scFV-library). Cation exchange chromatography (“CEX”) is a form of ion exchange chromatography (IEX), which is used to separate molecules based on their net surface charge. Cation exchange chromatography, more specifically, uses a negatively charged ion exchange resin with an affinity for molecules having net positive surface charges. Cation exchange chromatography is used both for preparative and analytical purposes and can separate a large range of molecules from amino acids and nucleotides to large proteins. Here, we focus on the preparative cation exchange chromatography of proteins. CEX can be performed under reducing and non-reduced conditions.

Mass spectrometry (“MS”) is an analytical technique that is used to measure the mass-to-charge ratio of ions. The results are presented as a mass spectrum, a plot of intensity as a function of the mass-to-charge ratio

The disclosure of each reference set forth herein is incorporated herein by reference in its entirety.

The present invention is further illustrated by the following Examples:

### EXAMPLES

ModVec is designed with extreme flexibility using a modular “slot-based” approach to assemble one or more expression cassettes in a variety of possible arrangements while allowing combinatorial exploration of sequence diversity within each slot. The Modvec system is divided into slots, where each slot can contain a DNA sequence with unique Golden Gate overhangs. This DNA sequences can be vector elements, including elements that are required for vector replication (ColE Ori,pMB1 Ori . . . ), antibiotic resistance (Amp, Kan, Crm . . . ), elements that are needed for viral infection (LTR), transposition (ITR), episomal replication (OriP),targeted integration (loxP, Frt, attB/P), and elements for mammalian gene expression (e.g., promoters, enhances, internal ribosomal entry sites (IRES), self-processing viral 2A peptide, polyA signal, control sequence, insulator sequences including MARs and UCOEs, etc).

As proof of principle, ModVec was first utlilized to assemble complex mammalian expression vectors. Two ModVec backbones were generate including pMVP5 which can be used in the common mammalian cell lines used in Research (HEK, CHO) and the vector backbone which was designed specifically to suit Amgen's manufacturing CHO cell lines. These vector backbones contain only minimal vector elements that are required for replication and antibiotic selection in E. coli for plasmid maintenance.

In the first experiment, the ModVec concept was tested by assembling a vector with functional DNA sequence in every slot: a 14-slot (including vector backbone), 8 kb DNA assembly. It contained three ECs; EC1 and EC2 were bicistronic and EC3 was monocistronic. It had three different promoters to drive expression of 5 coding sequences. It has been shown that using circular plasmids as the input for GG reactions increases efficiency especially for multi-piece golden gate assembly (Potapov, Ong et al. 2018). Therefore “parts” vectors were generated in which vector elements that were expected to befrequently re-used in each slot were cloned into pGG_Cm_BsaI vector first using GG with BsaI and confirmed by Sanger sequencing. In this process, the “parts library” of vector elements such as promoters, IRESs, polyAs, control sequence, insulators, etc with ModVec-suitable overhangs was built up in a format that will be easy to store and re-fill at need. Using a combination of circular “parts” and synthesized linear DNA, the first 14-slot assembly used 20 ul GG reaction with 1 ul of each vector element (at concentration between 40-70 n g/ul). pMVP5 was used as vector backbone. The GG reaction was transformed into E. coli and 32 colonies were picked for Sanger Sequencing. The design 8 KB DNA vector was successfully obtained; however, the efficiency of correct assembly was only 3.125% (1 of 32 picked colonies correct). Internal as well as external data (Kanigowska, Shen et al. 2016) showed that GG efficiency can be higher with smaller reaction volume (miniature GG). 14-slot assembly was then performed at smaller reaction volumes, 2 ul, 1 ul and 0.5 ul using an ECHO acoustic liquid handler that enables accurate liquid transfer at these very small volumes. These reactions were a proportional scale down from the initial 20 ul reaction. The miniature GG increased the efficiency of this 14-piece ModVec assembly to 16% (13 of 78 colonies correct).

By careful analysis of both the successes and failures of the first POC ModVec assemblies, it was determined that a specific set of 11 GG overhangs were always correctly ligated in this context. It was then tested if using only this set of overhangs could increase ModVec efficiency. ModVec was used to assemble a 10-slot assembly with these optimized overhangs in a 1 ul GG reaction. It was found that over 95% of the clones contained correctly assembled vectors (548 of 576 colonies correct).

The 95% efficiency of assembly obtained under optimized ModVec design and conditions enables one-tube construction of an expression vector library. The functionally similar vector elements that had same overhangs together are mixed to create “slot libraries.”. 2 different promoters to drive 12 CDSs in EC1 were combined with 3 different promoters to drive selection marker expression EC3, including the bridge that eliminates EC2, which through combinatorial assembly could result in 72 different expression vectors. After transformation, 350 colonies were picked, and after sequencing verification a total of 312 contigs were generated. These 312 contigs matched 69 out of 72 possible vectors, demonstrating low sequence-dependent biases in Modvec assembly and indicating that a library strategy is possible with this system.

### Materials and Methods

### Construction of Vectors

All DNA fragments including vector elements and antibody LCs and HCs were synthesized by Twist Bioscience or PCR amplified from existing templates and cloned into vector backbones using standard a Golden Gate assembly protocol (Engler et al., 2008). To generate part vectors, each Golden Gate reaction contained 2 μL of DNA fragments (5 ng/μL) and 2 μL of pGG vector (20 ng/μL), 1 μL of FastDigest buffer (Thermo Fisher, B64) with 5 μM ATP (Thermo Fisher, R0441), 0.5 μL T4 ligase (Thermo Fisher, EL0014) and 0.5 μL of BsaI (Thermo Fisher, ER0291), and 3 μL H2O. The Golden Gate reaction was run at 37° C. for 2 mins and 16° C. for 3 mins for 15 cycles on thermal cycler. A final 5 min incubation at 85° C. was performed to deactivate all enzymes. The miniaturized Golden Gate reaction and one pot vector library cloning reaction was set up using ECHO 525 liquid handler (Labcyte) to perform nanoliter scale liquid transfer. The volume of each DNA fragment and vector backbone was proportionally scaled down for the miniaturized Golden Gate reaction. For ModVec assembly, 15 μL Golden Gate reaction mixture including 2 μL of pGG part vectors (40-70 ng/μL), 2 μL of expression vector backbone (50 ng/μL), 1.5 μL of FastDigest buffer (Thermo Fisher, B64) with 5 μM ATP (Thermo Fisher, R0441), 0.75 μL T4 ligase (Thermo Fisher, EL0014) and 0.75 μL of BsmBI (Thermo Fisher, FD0454), and a variable amount of H₂O to make up 15 μL, was mixed using a TECAN liquid handler. The 37° C. incubation was extended to 3 mins and the 16° C. incubation to 5 min for each Golden Gate cycle and extended the cycle number to 20 for ModVec assembly. Plasmid DNA was prepared using a Qiagen miniprep Kit (Qiagen, 27104). After sequencing confirmation, plasmid DNA coding HC and LC were mixed at defined ratios.

Cell culture and Protein Expression

To generate stable cell pools through random genomic integration, 25 μg DNA were electroporated into our internal proprietary suspension Chinese Hamster Ovary cells (CHO) using the Bio-Rad Gene Pulser® Xcell Electroporation Systems. After electroporation, the entire transfection was seeded in proprietary recovery medium. Every 2-3 days until recovery, viable cell density and viability were monitored using a VI-CELL® counter (Beckman Coulter) and media was exchanged. Recovery was defined as >85% viability by VI-CELL®. Recovered cells were used to seed 50-mL fed-batch productions in shake flasks, which were harvested after 10 days. To generate stable cell pools using piggyBac transposase-based expression vectors, 4 μg DNA were used to transfect a proprietary suspension CHO cell with glutamine synthetase knocked out (CHO GS KO) using Lipofectamine LTX (Thermo Fisher, 11668030) at 4×10⁶ viable cells per mL. The transfected cells were transferred 48 to 72 hours post-transfection to selection media with methionine sulfoximine (MSX). Recovered cells were used to seed 4-mL fed batch productions in 24-well culture blocks at 1×10⁶ cells per mL, which were harvested after 10 days. During production, viable cell density and viability were monitored using a VI-CELL® counter (Beckman Coulter) and media was exchanged at day 3, 6, and 8. At day 10, viable cell density and viability were measured and the conditioned media from these batch productions were used to determine titer by ForteBio OCTET® Red equipped with Protein A biosensors.

### High Throughput Protein Purification

A KingFisher® Flex system (Thermo Fisher) with magnetic ProA beads (GE Life Sciences) was used to purify protein as previously described (Gong et al., 2021). Briefly, one day before harvest, 100 μL magnetic ProA beads were added to 4 mL CHO GS KO cultures. The beads were collected and subjected to Kingfisher purification with a 24-probe magnetic head. After washing 3 times with phosphate-buffered saline, and 2 times with Milli-Q water, proteins were eluted with 500 μL of 100 mM sodium acetate at pH 3.6 for 10 mins, and then neutralized by addition of 10 μL of 3 M Tris, pH 11.0.

### Large Scale ProA Purification Followed by Cation Exchange Chromatography

Proteins expressed in 50-mL CHO cultures were purified as previously described (Gong et al., 2021; Yoo et al., 2014) using ProA affinity capture (1 mL HiTrap MabSelect SuRe, GE Life Sciences, catalog #GE11-0034-93), eluted with 100 mM sodium acetate, pH 3.6 followed immediately by buffer exchange into 10 mM sodium acetate, 150 mM NaCl, pH 5.2 using a 5 mL HiTrap Desalting column (GE Life Sciences, catalog #GE17-1408-01).

Cation exchange chromatography was performed as previously described (Gong et al., 2021). Briefly, 1.5-1.8 mL ProA-purified samples were diluted with 20 mL of 20 mM MES, pH 6.2 and loaded onto a 1-mL cation exchange column (SP-HP HiTrap, GE Life Sciences, catalog #GE29-0513-24) at 1 mL/min. After washing the column with 8 column volumes of the same buffer at 1 mL/min, the proteins were eluted with a linear 0-400 mM NaCl gradient over 40 column volumes at 0.4 mL/min. Fractions of 90% or higher purity as determined by size exclusion chromatography (SEC) were pooled and their concentration was determined using a Multiscan GO microplate reader (Thermo Fisher) as previously described (Winters et al., 2015). The final pooled samples were analyzed by SEC, non-reducing microcapillary electrophoresis (nrMCE) and liquid chromatography-mass spectrometry (LC-MS).

### Non-Reducing Microcapillary Electrophoresis and Size Exclusion Chromatography

Product quality of purified materials were analyzed using reducing and non-reducing microcapillary electrophoresis and SEC as previously described (Gong et al., 2021; Guo et al., 2021). For nrMCE, 6 μL of protein was mixed with 21 μL of sample buffer (8.4 mM TrisHCl pH 7.0, 7.98% glycerol, 2.38 mM EDTA, 2.8% SDS, and 2.4 mM iodoacetamide), heated at 85° C. for 10 min, and then analyzed using a Caliper LabChip GXII Touch instrument (PerkinElmer). For analytical SEC, protein samples were analyzed on an Acquity® HPLC instrument (Waters) using a BEH column (200 Å, 1.7 micron, 4.7×300 mm) with 100 mM sodium phosphate pH 6.9, 50 mM NaCl, 7.5% ethanol as the running buffer at 0.45 mL/min flow rate.

### Liquid Chromatography-Mass Spectrometry and Rapid Fire-Mass Spectrometry

Intact non-reducing LC-MS was performed to determine MW of purified samples as previously reported in (Campuzano et al., 2019; Spahr et al., 2018). An aliquot of 30 μL of 1% trifluoroacetic acid (TFA) was added to 30 μL of each purified sample. Next, approximately 15 μg of each sample was injected on an Agilent 1290 UPLC with the column effluent directly coupled to an Agilent 6224 electrospray time of flight (ESI-TOF) mass spectrometer (Agilent Technologies). Chromatographic separation utilized a Zorbax® RRHD 300SB-C8 2.1×50 mm, 1.8 μm particle size ultra-performance liquid chromatography (UPLC) column (Agilent Technologies). The column was heated to 70° C. with a flow rate of 0.5 mL/min. Chromatographic solvents of aqueous “A” (0.1% TFA in H₂O) and organic “B” (0.1% TFA in 90% n-propanol) were used. The gradient used was isocratic at 80% A/20% B for 4 min, 28% A/72% B for 2 min, 10% A/90% B for 0.5 min, and finally 5% A/95% B for 0.5 min. The MS method scans m/z [1000-7000] acquiring 0.7 spectra/sec. The resulting spectra were summed then deconvoluted using either the Agilent Mass Hunter Qualitative Analysis software (Version B.07.00) or the Intact Program module from Protein Metrics (PMI Intact).

RapidFire-MS system was used on samples from the high throughput vector configuration library screening. An equal volume of 0.1% w/w formic acid was added to 50 uL of the sample supplied and 20 uL of this solution was injected on the RapidFire-MS for analysis. The SPE cartridge was a 4-uL PLRP 1000 Å cartridge/column. Mobile phases were 10% n-propanol containing 0.1% formic acid and 90% n-propanol containing 0.1% formic acid. All data was processed using PMI Intact.

### Results

### Modular Vector (ModVec) Platform for High Throughput Assembly of Complex Vectors

To enable high throughput vector engineering, a GG assembly-based mammalian modular vector (ModVec) system was developed to build diverse expression vectors and vector libraries for recombinant protein production. ModVec enables high throughput construction of both simple and complex vector designs to support optimization of expression vectors for individual molecules, for specific large-molecule modalities, and/or for different expression hosts.

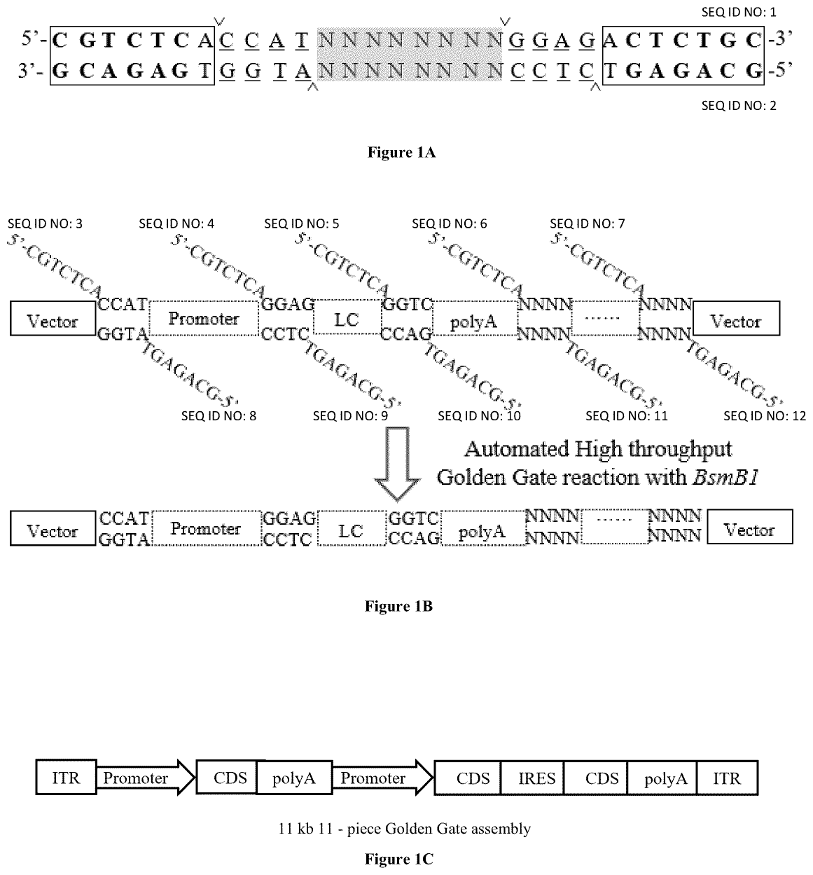

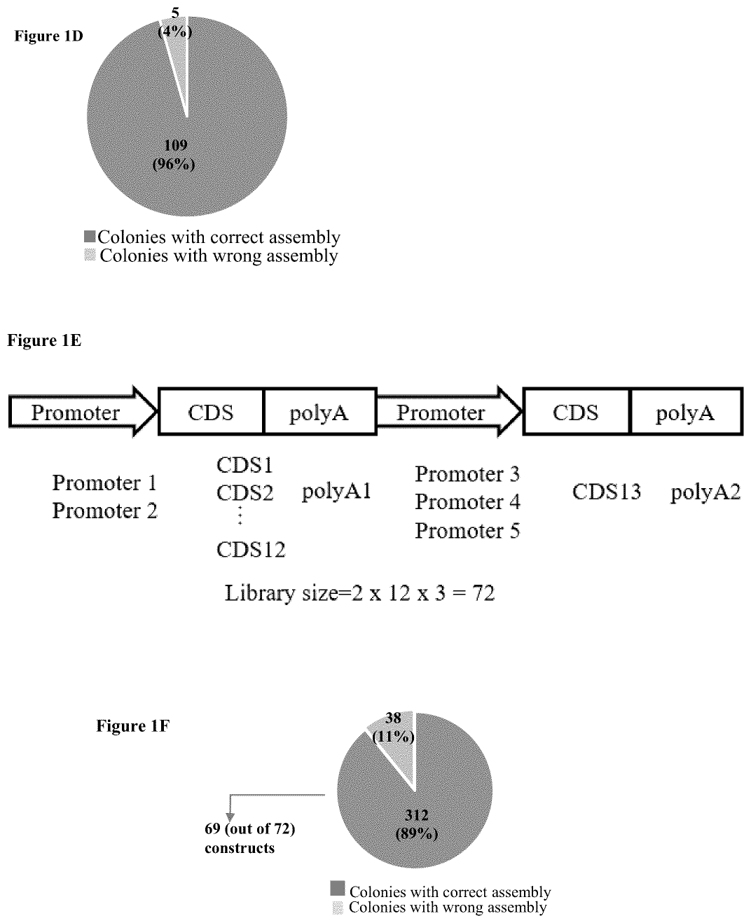

ModVec is designed with extreme flexibility to assemble one or more expression cassettes in a variety of possible arrangements while allowing combinatorial exploration of sequence diversity within each module. Each module contains a DNA sequence, or libraries of DNA sequences, with carefully designed GG overhangs (FIG. 1B). These DNA sequences can be vector elements commonly used in recombinant expression such as sequences that are required for vector replication (origin of replication) and antibiotic resistance, elements that are needed for viral infection, transposition, episomal replication, targeted integration, or elements commonly used for mammalian gene expression. These DNA sequences are flanked by GG adaptors (boxed region in FIG. 1A) that contain, for example, BsmBI restriction sites, and they can be generated through DNA synthesis or PCR amplification. A set of complementary, non-promiscuous four-bp overhangs, flanked by BsmBI restriction sites, was designed to enable seamless and unidirectional assembly of DNA elements into rational expression cassettes (FIG. 1B).

The ModVec concept was first tested by assembling 11 kb, 11-module bicistronic constructs in a 15 μL GG reaction (FIG. 1C). 19 constructs were assembled, and six colonies for each construct were inoculated for sequence verification. All 19 bicistronic constructs were successfully assembled through one round of cloning. Over 95% (109/114) of the clones contained correctly assembled vectors (FIG. 1D). These results demonstrate that ModVec is an efficient approach for assembly of large, complex pieces of DNA when the set of GG overhangs are compatible (i.e., non-promiscuous with each other). The 95% efficiency of correct assembly obtained with optimized ModVec enables one-tube construction of an expression vector library. The functionally similar vector elements that had same overhangs were mixed together to create “module libraries” (FIG. 1E). As a demonstration of single-tube vector engineering libraries, two different promoters driving 12 coding sequences (CDS 1 to CDS 12) were combined with three different promoters driving CDS 13 expression, which through combinatorial assembly could result in 72 different expression vectors. A miniaturized GG reaction was set up using the ECHO liquid handler which has been shown to increase GG assembly efficiency (Kanigowska et al., 2016). 80 nL of each of the 25 parts were mixed in a 2.5 μL GG assembly reaction. After 30 cycles of GG assembly, the full volume was transformed and plated. After overnight incubation, around 1000 colonies grew on the agar plate, 350 of which were inoculated in liquid culture for sequence verification by Sanger sequencing. A total of 312 contigs were generated which matched to 69 out of 72 possible vectors (FIG. 1F).

These results demonstrate that ModVec is an efficient cloning platform for HT assembly of complex expression vectors and expression configuration library. Therefore, it is now possible to improve productivity and product quality of bsAbs through high throughput vector configuration screening to identify vector configurations that have balanced expression level of polypeptide chain in bsAbs. The ModVec cloning system was next used to explore different transcription modulation strategies to improve the productivity and product quality of complex multichain multispecific antibodies produced recombinantly in mammalian cells.

Impact of Promoters on Productivity of Two-Chain Monoclonal Antibodies (mAb) and Symmetric Bispecific Antibodies (bsAb)

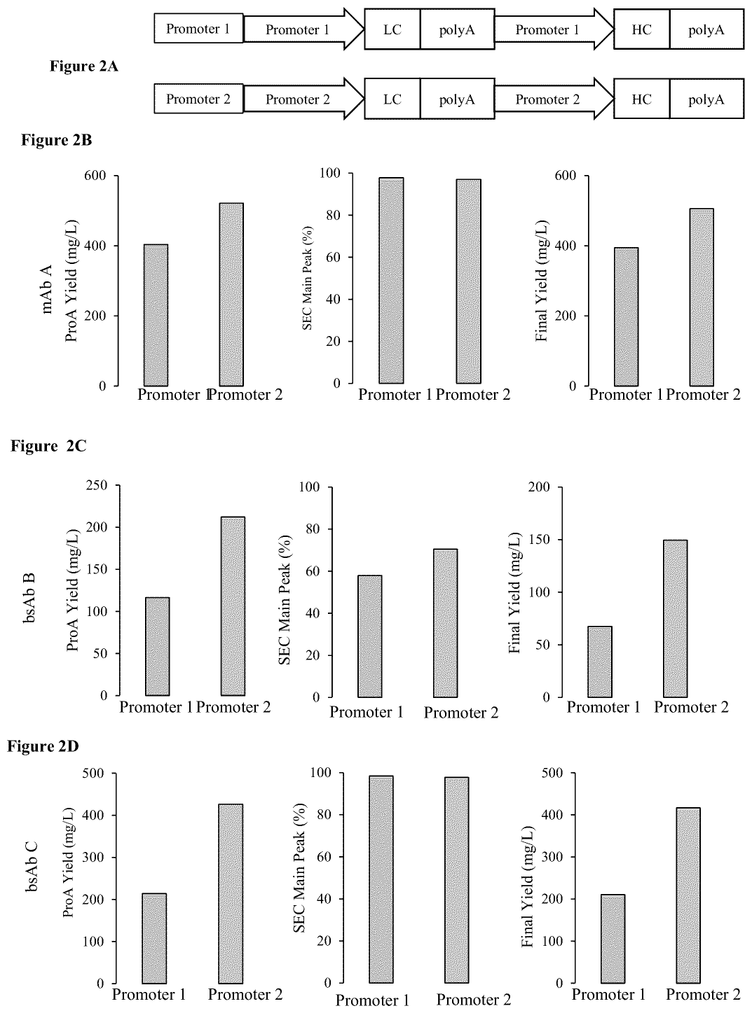

The impact of promoters tested on productivity of symmetric, two-chain molecules A, B, and C (FIGS. 2B-2D). Molecule A is a canonical monoclonal antibody, molecule B and C are symmetric bispecific antibodies. Bicistronic vectors were constructed in which the LC and HC genes for each molecule were driven by the same promoter (either Promoter 1 or Promoter 2) (FIG. 2A). Promoter 1 and Promoter 2 are all derived from huCMV promoter, but Promoter 2 is stronger than Promoter 1. A piggyBac transposase-based expression vector was used for efficient genomic integration and rapid generation of stable cell pools. Internal observations suggest that piggyBac transposase-based expression vectors have very high reproducibility, so one stable Chinese Hamster Ovarian (CHO) cell pool was generated for each configuration. Regardless of molecule type, pools transfected with constructs using Promoter 2 had higher yield after Protein A (ProA) affinity chromatography than pools transfected with constructs using Promoter 1 (FIG. 2B-2D). The ProA yield of molecule A was 403 mg/L in a pool transfected with constructs using Promoter 1 and was 521 mg/L in a pool transfected with constructs using Promoter 2, a 30% increase in yield by switching Promoter 1 to Promoter 2. More dramatic increases were observed for molecules B and C, where switching Promoter 1 to Promoter 2 led to an 83% and 100% increase in ProA yield, respectively. The cell recovery and growth characteristics were also comparable between pools transfected with all constructs. For these three symmetric molecules, altering the promoter strength of both HC and LC genes together did not have significant impact on product quality as measured by percentage of main species in size exclusion chromatography (SEC % main peak, FIG. 2B-2D, middle panel). As a result, an increase of 28%, 122%, and 96% in the final yield of molecule A, B, C was achieved by using Promoter 2. More dramatic increases in productivity were observed for the molecules that initially had lower yields (molecules B and C). Results indicate that modulation of promoter strength is an effective approach to improve the productivity of mAbs and bsAbs, and further that it might be most effectively applied to difficult-to-express proteins.

Vector engineering strategies were next expanded to improve both productivity and product quality of Hetero-IgG, a more complex four-chain bsAb.

Improvements in Product Quality and Productivity for Hetero-IgG through Chain Balance

Hetero-IgG is the most common bispecific format because of its antibody-like structure (Labrijn et al., 2019), but it is also more challenging to manufacture than mAbs because of the possibility of multiple product-derived impurities caused by incorrect LC-HC and HC-HC pairing (Brinkmann & Kontermann, 2017). Many protein engineering strategies such as Charge Pair Mutation (CPM) (Dillon et al., 2017; Gunasekaran et al., 2010), Knobs-into-Holes (KiH) (Ridgway et al., 1996), strand-exchange engineered domain (SEED) (Davis et al., 2010) and common light chain (Krah et al., 2017; Shiraiwa et al., 2019) have been used to favor correct chain pairing and prevent incorrect chain pairing, thereby improving the manufacturability of Hetero-IgG. However, differences in the expression level of each polypeptide chain comprising the Hetero-IgG in mammalian cells can lead to formation of product related impurities and reduce the yield of correct bsAb. For example, in stable CHO pools of a molecule, Hetero-IgG-D, that were transfected with our default expression vectors, up to 60-fold higher production of LC1 relative to LC2 was observed and secretion of correspondingly high amounts of mis-paired species with two copies of LC1(Guo et al., 2021). It was hypothesized that the yield of correctly paired Hetero-IgG relative to mis-paired product-related impurities could be improved by balancing the expression of each polypeptide chain closer to a 1:1:1:1 ratio through vector engineering. Informed by our success with two-chain molecules, a vector engineering approach of balancing the polypeptide chain expression using promoters of different strength was taken.

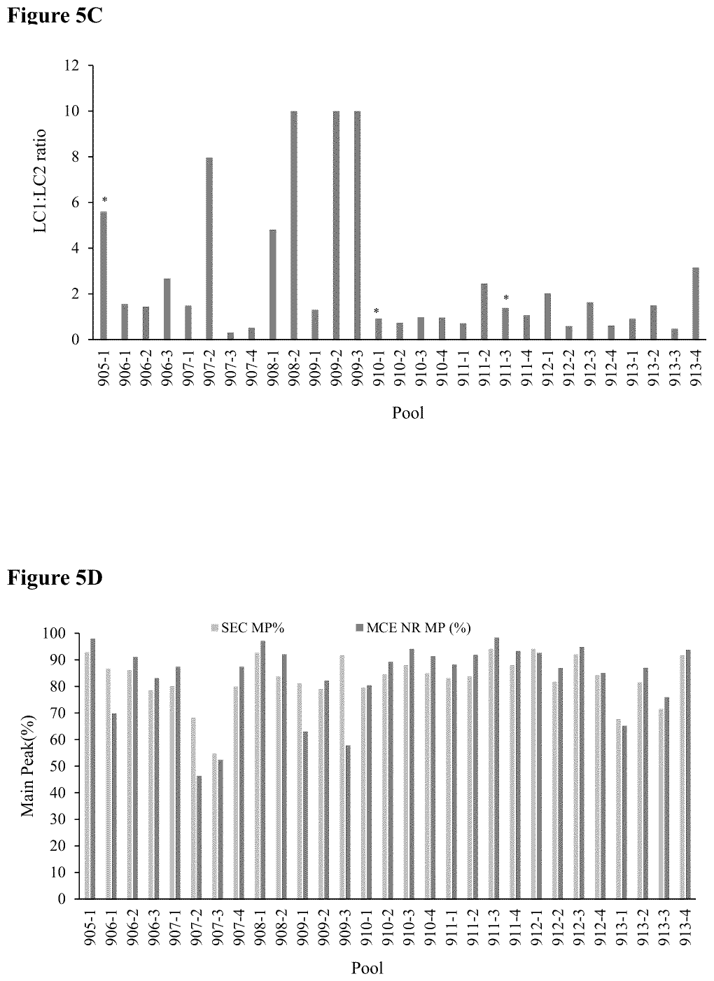

11 vector configurations for Hetero-IgG-D were constructed using ModVec. Each vector configuration contained two co-transfected bicistronic vectors in which the two polypeptide CDSs in that vector were driven either by the same or by different promoters (Promoter 1, Promoter 2, and Promoter 3, FIG. 5A). In some vector configurations, the position of LC or HC coding sequences were swapped within the bicistronic cassette (FIG. 5A Config 2). 4 transfections per vector configuration were carried out in CHO cells, and a total of 44 pools were generated. The stable pools were generated through random genomic integration with metabolic selection. Pools from different vector configurations had different recovery rates; of the 44 pools transfected, 15 did not recover. Those that failed to recover tended to be transfected with configurations in which LC1/LC2 and HC1/HC2 were in the same vector (Config 2 in FIG. 5A). In addition to differences in recovery of stable CHO pools, the vector configuration library demonstrated a wide range in yield after ProA affinity purification (FIG. 5B), in product quality measured by LC1:LC2 ratio (FIG. 5C), and in percentage of the peak for main species (% MP) in non-reducing microcapillary electrophoresis (nrMCE) and size exclusion chromatography (SEC) (FIG. 5D).

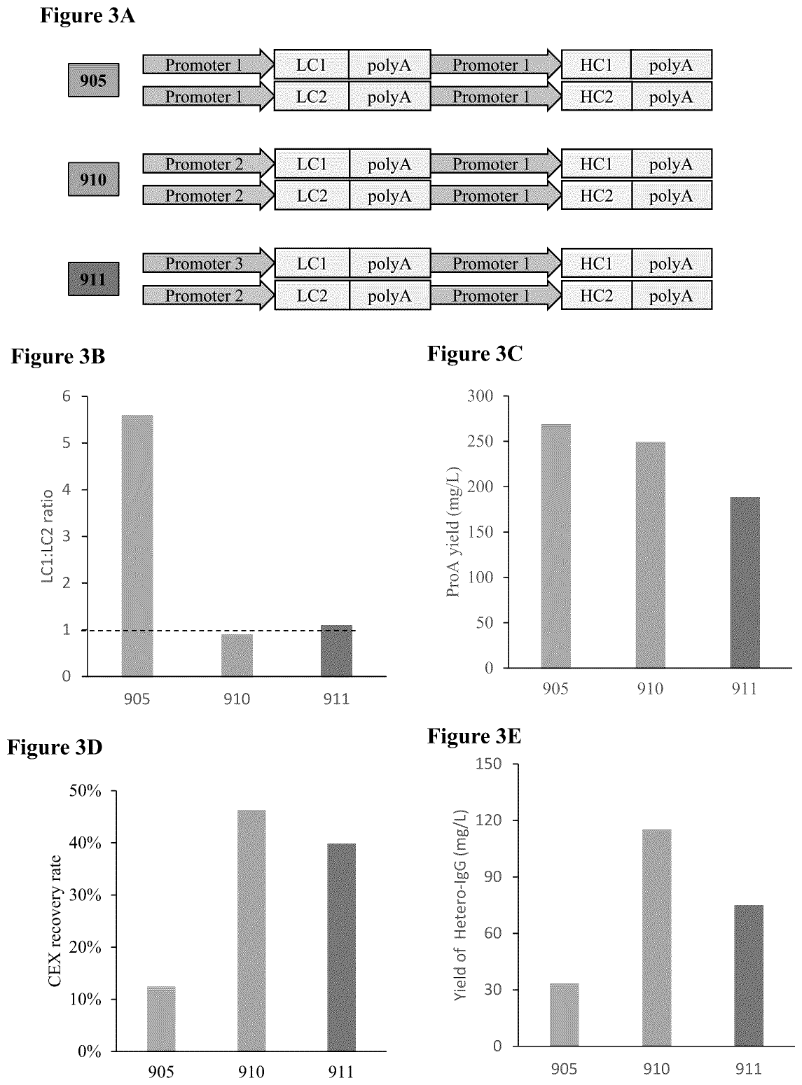

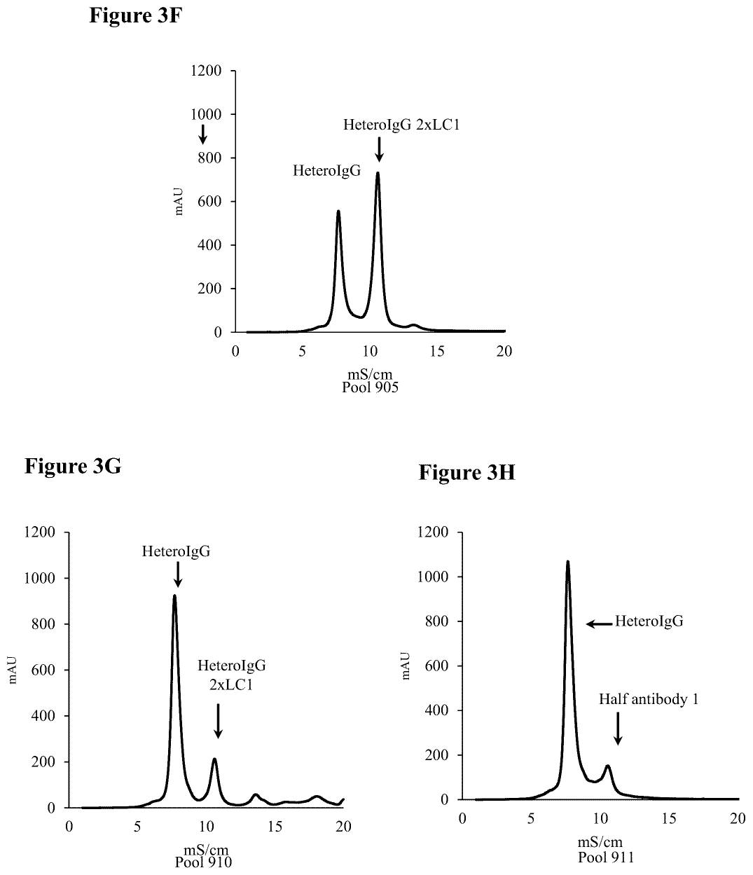

Three pools were selected for scaled up production and further analysis: two pools had LC1:LC2 ratio close to the ideal value of 1 (FIG. 3B, pools 910 and 911,) and one pool had a skewed LC1:LC2 ratio >5 (FIG. 3B, pool 905). The vector configurations used to generate these stable pools were illustrated in FIG. 3A. The conditioned media of these three scaled up pools was purified with ProA affinity capture followed by cation exchange (CEX) using Sepharose SP resin. All three pools had similar ProA yield (FIG. 3C). The recovery rate of the main Hetero-IgG-D species from Pool 905 after cation exchange was 12%, while pools 910 and 911 had recovery rates of 46% and 40% respectively (FIG. 3D). Consequently, the yield of correctly assembled Hetero-IgG-D after cation exchange was higher for pools 910 and 911 (115 and 75 mg/mL, respectively) than for pool 905 (34 mg/L) (FIG. 3E).

In addition to producing different relative levels of the main species, the pools transfected with different vector configurations also had a different profile of product-related impurities, as shown in the cation exchange chromatograms (FIG. 3F-3H). The main species in ProA-purified samples of pool 905 was identified by mass spectrometry as a mis-paired hetero-tetramer with 2 copies of LC1 (2× LC1, i.e., LC1:HC1:LC1:HC2, major peak in FIG. 3F), while the correctly assembled Hetero-IgG-D was the minor species (minor peak in FIG. 3F). This explains the low purification yield and recovery rate of pool 905 (FIG. 3D-3E) and is consistent with the high LC1:LC2 ratio (FIG. 3B). This also indicates that the expression level of LC1 and LC2 was out of balance when Promoter 1 was used to drive expression of both LCs. We did not observe antibody-like homodimers from pool 905, which would result from HC1:HC2 mispairing; the main issue with the CHO pool transfected with this vector configuration was LC-HC mispairing. When the promoter driving both LC1 and LC2 was switched to Promoter 2 (pool 910, FIG. 3A), the 2× LC1 became the minor species and correctly assembled Hetero-IgG-D became the major species (FIG. 3G) in ProA purified materials, which resulted in the higher recovery of main species after cation exchange (FIG. 3D-3E). It is interesting that changing both LC promoters to Promoter 2 brings expression of LC1 and LC2 into balance. We previously reported that LC2 secreted poorly from mammalian cells (Guo et al., 2021). It is possible that switching both LC promoters to Promoter 2 had a greater impact on the “expression-deficient” LC2, leading to a more balanced LC1:LC2 ratio. We have transfected >10 replicate CHO pools with the promoter arrangement used to generate pool 905 and consistently observed high LC1:LC2 ratio (5-60) and high levels of 2× LC1 species. Furthermore, in pool 911, when the promoter of LC1 is changed to Promoter 3, which has transcriptional strength intermediate between that of Promoter 1 and Promoter 2, while the LC2 promoter is unchanged (Promoter 2), the correctly assembled Hetero-IgG-D remained the main species. However, the dominant impurity was changed to half antibody 1 (LC1:HC1) instead of 2× LC1 (FIG. 3H). The change in the main impurity from 2× LC1 in pools 905 and 910 to half antibody 1 in pool 911 may be a result of changes in the LC gene promoters impacting expression of both LC and HC genes from the bicistronic cassettes.

### High Throughput Vector Engineering for an Asymmetric, Three-Chain Hetero-IgG-Based Trispecific Molecule

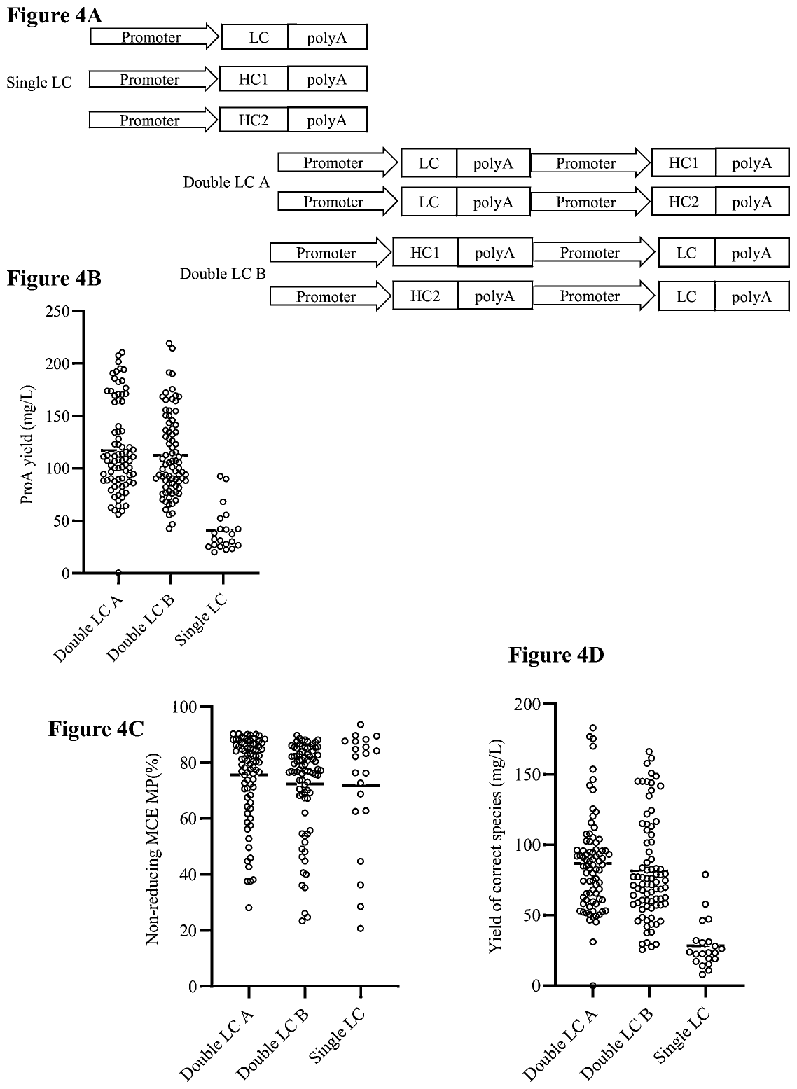

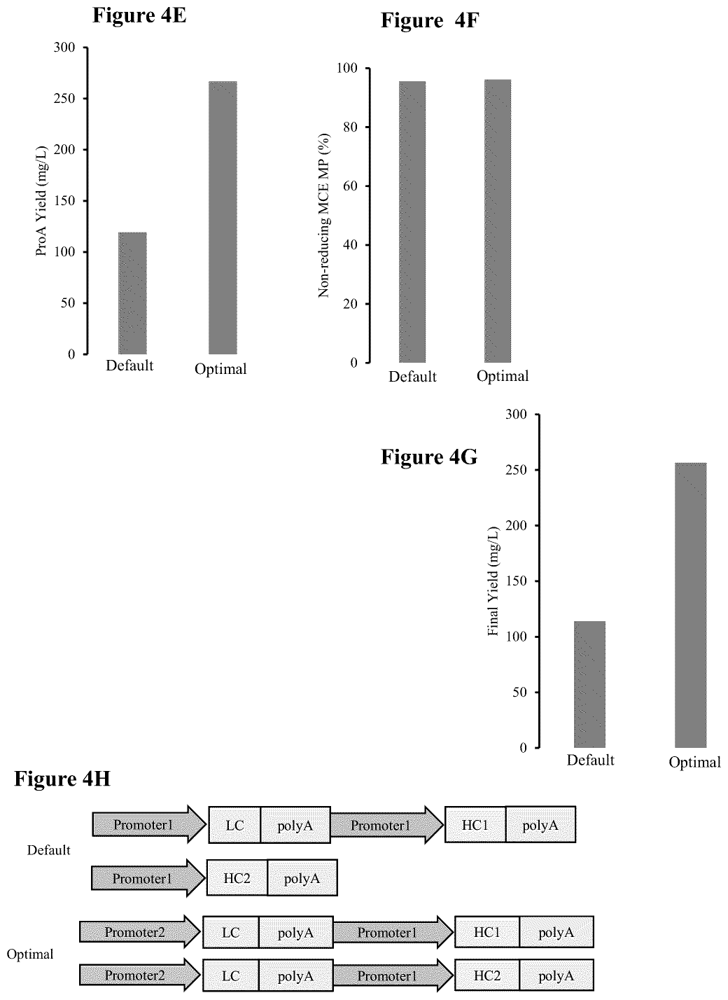

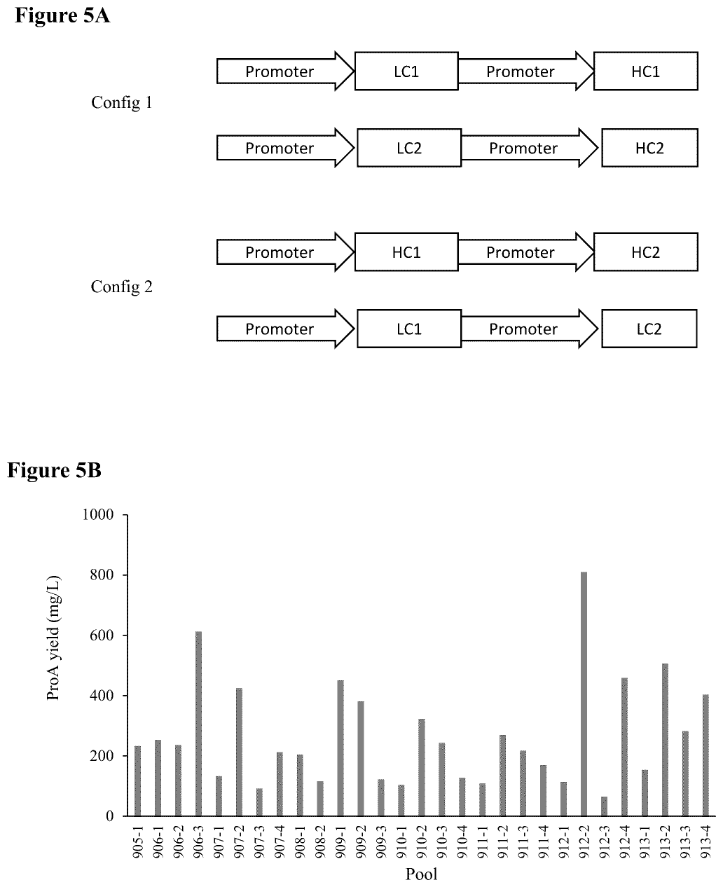

It was next tested if high-productivity vectors for a challenging three-chain asymmetric trispecific antibody E (tsAb E) could be engineered. In transient transfection tests, tsAb E was produced at titers 5 to 10-fold lower than expected levels for mAbs. Inspired by success of the 11-vector configuration screening for a Hetero-IgG, it was decided to apply a vector engineering approach to improve the productivity of tsAb E but included a larger number of vector configurations in the library design. It was conjectured that testing more strategies to modulate polypeptide chain expression would increase the probability of finding configurations with stoichiometrically optimal ratios for all three polypeptide chains of asymmetric tsAb E. In addition to promoters of different strengths, gene dosage (LC gene copy number) was varied, and the cistronic arrangement of LC and HC genes as variables in the library design. The final library comprised 189 vector configurations that included 27 monocistronic vectors with a single copy of the LC gene (single LC), 81 bicistronic vectors with two copies of the LC gene in the first cistron of each vector (double LC A), and 81 bicistronic vectors with two copies of the LC gene in the second cistron of each vector (double LC B) (FIG. 4A). Three promoters, (Promoter 1, Promoter 2, and Promoter 3) were used to drive expression of each polypeptide chain in a combinatorial fashion; therefore, in this vector library design, genes in a bicistronic vector could optionally be driven by the same or different promoters.

CHO cells were transfected in 24-well culture plates using a proprietary high throughput expression system that relies on transposase-mediated integration with metabolic selection. One pool was generated for each vector configuration. All pools had comparable recovery and growth profiles (data not shown), which is common for this expression system. As shown in FIG. 4B, there was an order of magnitude difference in the productivity of this tsAb depending on vector configuration; the ProA yield of all pools ranged from 20 mg/L to 220 mg/L, which clearly demonstrated the power of vector configuration to influence yield. Pools transfected with double LC A (117.2±43.4 mg/L), or double LC B (112.6±39.2 mg/L) configurations had higher average ProA yield than pools transfected with single LC configurations (40.7±20.73 mg/L) (student t-test, double LC A vs single LC p<0.0001, double LC B vs single LC p<0.0001; values expressed as mean±standard deviation). Double LC A pools and double LC B pools had comparable ProA yield (student t-test, double LC vs double LC B p=0.48). A preliminary assessment of product quality was done by running non-reducing microcapillary electrophoresis (nrMCE) on each ProA pool; the average percentages of the peak for main species (% MP) between pools generated from these three groups of vector configurations were comparable (FIG. 4C, double LC A MP: 75.6±14.9%, double LC B MP: 72.4±16.6%, single LC MP: 71.7±21.6%; double LC A vs double LC B p=0.19, double LC A vs single LC p=0.34, double LC B vs single LC p=0.88, student t-test). As a result, as shown in FIG. 4D the average yield of correctly assembled tsAb E was higher in pools generated from double LC A (88.7±33.6 mg/L) or double LC B (81.4±35.6 mg/L) configurations than that of pools generated from single LC configurations (28.5±16.8 mg/L). The yield of correct species was comparable between pools generated from double LC A and double LC B (double LC A vs double LC B p=0.48, double LC A vs single LC p<0.0001, double LC B vs single LC p<0.0001, Student t-test). These results show the impact of LC gene copy number on productivity of complex therapeutic molecules. This is consistent with previous findings that gene dosage dictated by gene copy numbers can have big impact on productivity and that excess LC is overall beneficial for productivity (Gonz lez et al., 2002).

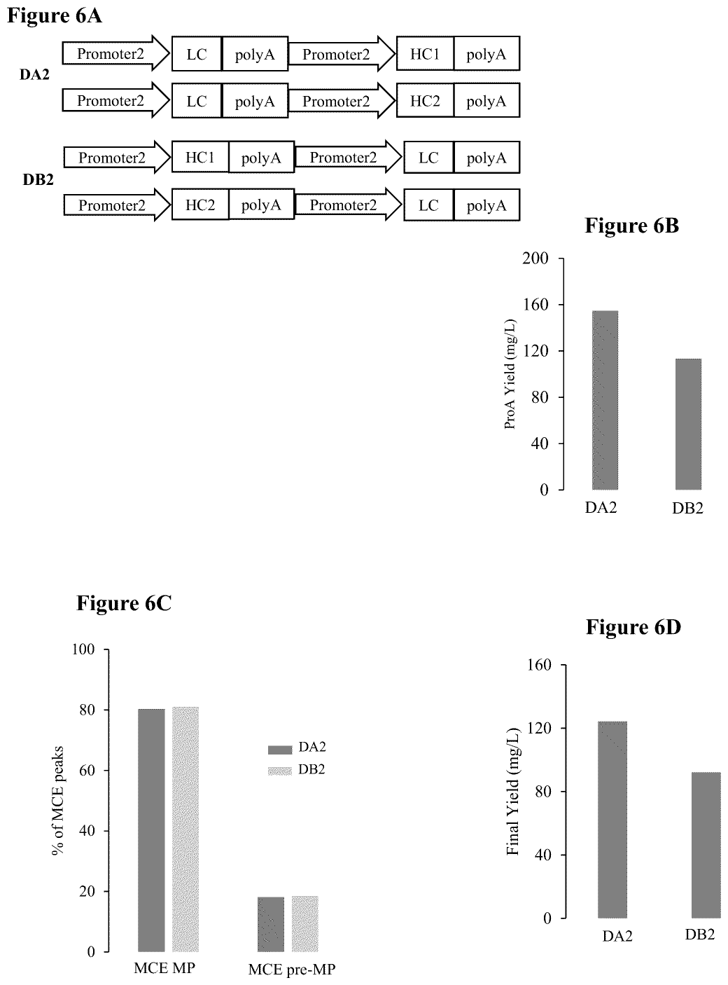

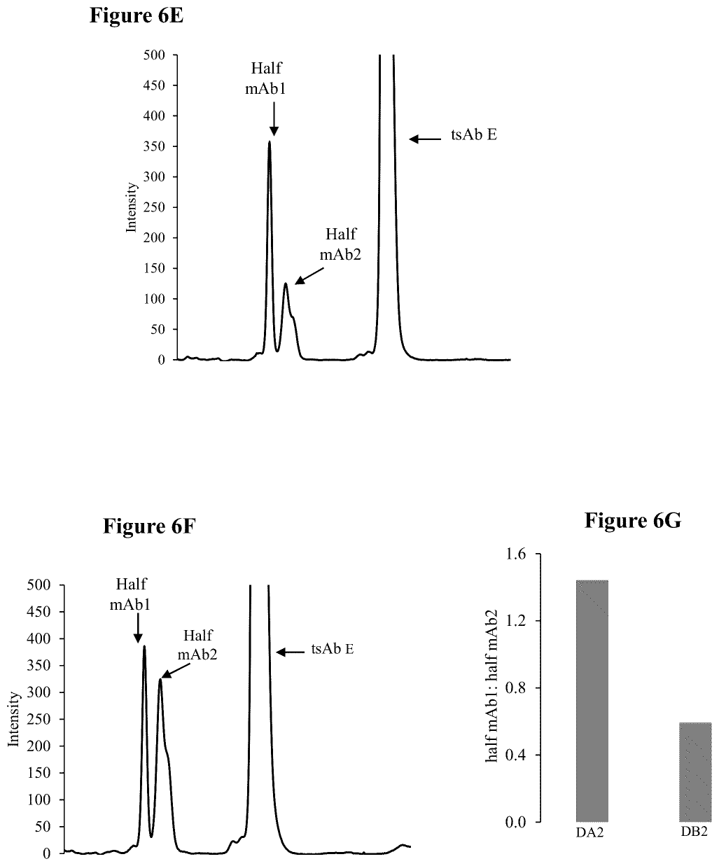

Since the performance of the double LC A group and double LC B group were comparable, the performance of individual configurations from these two groups was compared to understand the impact of gene position on productivity and product quality. Configurations DA2 (double LC A configuration with Promoter 2 driving the expression of all polypeptide chains) and DB2 (double LC B configuration with Promoter 2 driving the expression of all polypeptide chains) were chosen for further analysis (FIG. 6A). In DA2, the LC genes were in the first cistron and HC genes were in the second cistron while the position of LC genes and HC genes were swapped in DB2. As shown in FIG. 6B, the ProA yield of pool DA2 was 155 mg/L. Swapping the position of LC and HC genes by putting HC genes in the first cistron and LC genes in the second cistron decreased the overall productivity. The ProA yield of pool DB2 was 113 mg/L. This is consistent with the gene positional effect in multi-cistronic expression vectors where the expression levels of genes downstream of the first gene are generally reduced relative to the first gene's expression level (Eszterhas et al., 2002; Patel et al., 2021). In this case, lowering the expression of LC genes while increasing expression of HC genes lead to overall decrease in productivity, aligned again with the expectation that high level of LC expression is beneficial for overall productivity (Gonz lez et al., 2002). The product quality measured by nrMCE MP and pre-MPs was comparable between ProA samples purified from pools DA2 and DB2 (nrMCE MP, 80% for DA2 and 81% for DB2; nrMCE pre-MP, 18% for DA2 and 19% for DB2; FIG. 6C). Mass spectrometry confirmed that nrMCE MP was correctly assembled tsAb E and nrMCE pre-MPs were half mAbl (LC:HC1) and half mAb2 (LC:HC2, FIGS. 6E and 6F).

Even though the nrMCE pre-MP levels were comparable between ProA samples from pools DA2 and DB2, the relative abundance of half mAbl and mAb2 differed (FIG. 6E and 6F). The relative peak area of half mAb1 was 10.1 and half mAb2 was 7.4 in the nrMCE electropherogram of ProA-purified samples from pool DA2 (FIG. 6E). The relative peak area for half mAb1 was 5.8 and half mAb2 was 9.8for ProA-purified samples from pool DB2 (FIG. 6F). The half mAb1: half mAb2 relative peak ratio was calculated and the results are shown in FIG. 6G. The half mAb1: half mAb2 relative peak ratio was 1.4 in ProA-purified samples from pool DA2 and was 0.6 in ProA-purified samples from pool DB2. These results further demonstrate that LC or HC gene position in the expression vectors can significantly impact both productivity of main species and the profile of impurities produced from CHO cells. These results also support our hypothesis that combining promoters of different strength, gene copy number, and gene position is an effective approach to modulate expression levels of polypeptide chain and can increase the probability of identifying vector configurations that produce high productivity and product quality for multichain protein therapeutics

The top vector configurations were selected from the vector library screening to advance to cell line development (CLD) for tsAb E and compared these vectors with the default platform vector (shown in FIG. 4H). The default vector configuration uses Promoter 1 drive the expression of all polypeptide chains and combines a bicsitronic vector with LC gene in the first cistron and HC1 in the second cistron with a monocistronic vector containing HC2. In the contrast, the optimal configuration identified from the vector library consisted of two bicistronic vectors, each expressing a copy of the LC gene, in the first cistron with either HC1 or HC2 in the second cistron, with Promoter 2 driving the expression of both LC genes and Promoter 1 driving the expression of the HC1 and HC2 genes (FIG. 4H). Stable CHO pools generated from the default configuration produced tsAb E with a ProA yield of 119 mg/L, while the optimal configuration from the vector library yielded 267 mg/L after ProA, was an increase of 124% (FIG. 4E). The product quality measured by nrMCE was comparable between ProA-purified samples from both pools (FIG. 4F). As a result, the final yield of correctly assembled tsAb E was 114 mg/L in the default pool and 257 mg/L in the optimal pool (FIG. 4G). The optimal vector configuration identified through vector configuration library screening enabled 125% increase in productivity over our default vector configuration for this exciting and difficult to expression tsAb therapeutic.

### REFERENCES

Bhoskar, P., Belongia, B., Smith, R., Yoon, S., Carter, T., & Xu, J. (2013). Free light chain content in culture media reflects recombinant monoclonal antibody productivity and quality. Biotechnol Prog, 29(5), 1131-1139. https://doi.org/10.1002/btpr.1767
Blanco, N., Williams, A. J., Tang, D., Zhan, D., Misaghi, S., Kelley, R. F., & Simmons, L. C. (2020). Tailoring translational strength using Kozak sequence variants improves bispecific antibody assembly and reduces product-related impurities in CHO cells. Biotechnol Bioeng, 117(7), 1946-1960. https://doi.org/10.1002/bit.27347
Brinkmann, U., & Kontermann, R. E. (2017). The making of bispecific antibodies. MAbs, 9(2), 182-212. https://doi.org/10.1080/19420862.2016.1268307
Campuzano, I. D. G., Robinson, J. H., Hui, J. O., Shi, S. D., Netirojjanakul, C., Nshanian, M., . . . Bern, M. (2019). Native and Denaturing MS Protein Deconvolution for Biopharma: Monoclonal Antibodies and Antibody-Drug Conjugates to Polydisperse Membrane Proteins and Beyond. Anal Chem, 91(15), 9472-9480. https://doi.org/10.1021/acs.analchem.9b00062
Carver, J., Ng, D., Zhou, M., Ko, P., Zhan, D., Yim, M., . . . Hu, Z. (2020). Maximizing antibody production in a targeted integration host by optimization of subunit gene dosage and position. Biotechnol Prog, 36(4), e2967. https://doi.org/10.1002/btpr.2967
Cazier, A. P., & Blazeck, J. (2021). Advances in promoter engineering: Novel applications and predefined transcriptional control. Biotechnol J, 16(10), e2100239. https://doi.org/10.1002/biot.202100239
Davis, J. H., Aperlo, C., Li, Y., Kurosawa, E., Lan, Y., Lo, K. M., & Huston, J. S. (2010). SEEDbodies: fusion proteins based on strand-exchange engineered domain (SEED) CH3 heterodimers in an Fc analogue platform for asymmetric binders or immunofusions and bispecific antibodies. Protein Eng Des Sel, 23(4), 195-202. https://doi.org/10.1093/protein/gzp094
Deshaies, R. J. (2020). Multispecific drugs herald a new era of biopharmaceutical innovation. Nature, 580(7803), 329-338. https://doi.org/10.1038/s41586-020-2168-1
Dillon, M., Yin, Y., Zhou, J., McCarty, L., Ellerman, D., Slaga, D., . . . Carter, P. J. (2017). Efficient production of bispecific IgG of different isotypes and species of origin in single mammalian cells. MAbs, 9(2), 213-230. https://doi.org/10.1080/19420862.2016.1267089
Dong, E., Lam, C., Tang, D., Louie, S., Yim, M., Williams, A. J., . . . Misaghi, S. (2021). Concurrent transfection of randomized transgene configurations into targeted integration CHO host is an advantageous and cost-effective method for expression of complex molecules. Biotechnol J, 16(4), e2000230. https://doi.org/10.1002/biot.202000230
Eisenhut, P., Mebrahtu, A., Moradi Barzadd, M., Thalen, N., Klanert, G., Weinguny, M., . . . Rockberg, J. (2020). Systematic use of synthetic 5′-UTR RNA structures to tune protein translation improves yield and quality of complex proteins in mammalian cell factories. Nucleic Acids Res, 48(20), e119. https://doi.org/10.1093/nar/gkaa847
Engler, C., Kandzia, R., & Marillonnet, S. (2008). A one pot, one step, precision cloning method with high throughput capability. PLOS One, 3(11), e3647. https://doi.org/10.1371/journal.pone.0003647
Eszterhas, S. K., Bouhassira, E. E., Martin, D. I., & Fiering, S. (2002). Transcriptional interference by independently regulated genes occurs in any relative arrangement of the genes and is influenced by chromosomal integration position. Mol Cell Biol, 22(2), 469-479. https://doi.org/10.1128/MCB.22.2.469-479.2002
Fan, G., Wang, Z., Hao, M., & Li, J. (2015). Bispecific antibodies and their applications. J Hematol Oncol, 8, 130. https://doi.org/10.1186/s13045-015-0227-0
Gong, D., Riley, T. P., Bzymek, K. P., Correia, A. R., Li, D., Spahr, C., . . . Garces, F. (2021). Rational selection of building blocks for the assembly of bispecific antibodies. MAbs, 13(1), 1870058. https://doi.org/10.1080/19420862.2020.1870058
Gonz lez, R., Andrews, B. A., & Asenjo, J. A. (2002). Kinetic model of BiP- and PDI-mediated protein folding and assembly. J Theor Biol, 214(4), 529-537. https://doi.org/10.1006/jtbi.2001.2478
Gunasekaran, K., Pentony, M., Shen, M., Garrett, L., Forte, C., Woodward, A., . . . Yan, W. (2010). Enhancing antibody Fc heterodimer formation through electrostatic steering effects: applications to bispecific molecules and monovalent IgG. J Biol Chem, 285(25), 19637-19646. https://doi.org/10.1074/jbc.M110.117382
Guo, C., Chen, F., Xiao, Q., Catterall, H. B., Robinson, J. H., Wang, Z., . . . Hubert, R. (2021). Expression liabilities in a four-chain bispecific molecule. Biotechnol Bioeng, 118(10), 3744-3759. https://doi.org/10.1002/bit.27850
Ha, J. H., Kim, J. E., & Kim, Y. S. (2016). Immunoglobulin Fc Heterodimer Platform Technology: From Design to Applications in Therapeutic Antibodies and Proteins. Front Immunol, 7, 394. https://doi.org/10.3389/fimmu.2016.00394
Kaneyoshi, K., Kuroda, K., Uchiyama, K., Onitsuka, M., Yamano-Adachi, N., Koga, Y., & Omasa, T. (2019). Secretion analysis of intracellular “difficult-to-express” immunoglobulin G (IgG) in Chinese hamster ovary (CHO) cells. Cytotechnology, 71(1), 305-316. https://doi.org/10.1007/s10616-018-0286-5
Kanigowska, P., Shen, Y., Zheng, Y., Rosser, S., & Cai, Y. (2016). Smart DNA Fabrication Using Sound Waves: Applying Acoustic Dispensing Technologies to Synthetic Biology. J Lab Autom, 21(1), 49-56. https://doi.org/10.1177/2211068215593754
Krah, S., Schroter, C., Eller, C., Rhiel, L., Rasche, N., Beck, J., . . . Becker, S. (2017). Generation of human bispecific common light chain antibodies by combining animal immunization and yeast display. Protein Eng Des Sel, 30(4), 291-301. https://doi.org/10.1093/protein/gzw077
Kwaks, T. H., & Otte, A. P. (2006). Employing epigenetics to augment the expression of therapeutic proteins in mammalian cells. Trends Biotechnol, 24(3), 137-142. https://doi.org/10.1016/j.tibtech.2006.01.007
Labrijn, A. F., Janmaat, M. L., Reichert, J. M., & Parren, P. (2019). Bispecific antibodies: a mechanistic review of the pipeline. Nat Rev Drug Discov, 18(8), 585-608. https://doi.org/10.1038/s41573-019-0028-1
Ma, J., Mo, Y., Tang, M., Shen, J., Qi, Y., Zhao, W., . . . Qian, C. (2021). Bispecific Antibodies: From Research to Clinical Application. Front Immunol, 12, 626616. https://doi.org/10.3389/fimmu.2021.626616
Magistrelli, G., Pontini, G., Poitevin, Y., Malinge, P., Bourguignon, J., Gauye, F., . . . Fischer, N. (2018). Tuning Relative Polypeptide Expression to Optimize Assembly, Yield and Downstream Processing of Bispecific Antibodies. Antibodies (Basel), 7 (3). https://doi.org/10.3390/antib7030029
Mathias, S., Wippermann, A., Raab, N., Zeh, N., Handrick, R., Gorr, I., . . . Otte, K. (2020). Unraveling what makes a monoclonal antibody difficult-to-express: From intracellular accumulation to incomplete folding and degradation via ERAD. Biotechnol Bioeng, 117(1), 5-16.https://doi.org/10.1002/bit.27196
McBurney, M. W., Mai, T., Yang, X., & Jardine, K. (2002). Evidence for repeat-induced gene silencing in cultured Mammalian cells: inactivation of tandem repeats of transfected genes. Exp Cell Res, 274(1), 1-8. https://doi.org/10.1006/excr.2001.5443
Neves, D., Vos, S., Blank, L. M., & Ebert, B. E. (2019). Pseudomonas mRNA 2.0: Boosting Gene Expression Through Enhanced mRNA Stability and Translational Efficiency. Front Bioeng Biotechnol, 7, 458. https://doi.org/10.3389/fbioe.2019.00458
Patel, Y. D., Brown, A. J., Zhu, J., Rosignoli, G., Gibson, S. J., Hatton, D., & James, D. C. (2021). Control of Multigene Expression Stoichiometry in Mammalian Cells Using Synthetic Promoters. ACS Synth Biol, 10(5), 1155-1165. https://doi.org/10.1021/acssynbio.0c00643
Rajendra, Y., Peery, R. B., Hougland, M. D., Barnard, G. C., Wu, X., Fitchett, J. R., . . . Demarest, S. J. (2017). Transient and stable CHO expression, purification and characterization of novel hetero-dimeric bispecific IgG antibodies. Biotechnol Prog, 33(2), 469-477. https://doi.org/10.1002/btpr.2414
Ridgway, J. B., Presta, L. G., & Carter, P. (1996). ‘Knobs-into-holes’ engineering of antibody CH3 domains for heavy chain heterodimerization. Protein Eng, 9(7), 617-621. https://doi.org/10.1093/protein/9.7.617
Romanova, N., & Noll, T. (2018). Engineered and Natural Promoters and Chromatin-Modifying Elements for Recombinant Protein Expression in CHO Cells. Biotechnol J, 13 (3), e1700232. https://doi.org/10.1002/biot.201700232
Schaefer, W., Volger, H. R., Lorenz, S., Imhof-Jung, S., Regula, J. T., Klein, C., & Molhoj, M. (2016). Heavy and light chain pairing of bivalent quadroma and knobs-into-holes antibodies analyzed by UHR-ESI-QTOF mass spectrometry. MAbs, 8(1), 49-55. https://doi.org/10.1080/19420862.2015.1111498
Schlatter, S., Stansfield, S. H., Dinnis, D. M., Racher, A. J., Birch, J. R., & James, D. C. (2005). On the optimal ratio of heavy to light chain genes for efficient recombinant antibody production by CHO cells. Biotechnol Prog, 21(1), 122-133. https://doi.org/10.1021/bp049780w
Shiraiwa, H., Narita, A., Kamata-Sakurai, M., Ishiguro, T., Sano, Y., Hironiwa, N., . . . . Igawa, T. (2019). Engineering a bispecific antibody with a common light chain: Identification and optimization of an anti-CD3 epsilon and anti-GPC3 bispecific antibody, ERY974. Methods, 154, 10-20. https://doi.org/10.1016/j.ymeth.2018.10.005
Spahr, C. S., Daris, M. E., Graham, K. C., Soriano, B. D., Stevens, J. L., & Shi, S. D. (2018). Discovery, characterization, and remediation of a C-terminal Fc-extension in proteins expressed in CHO cells. MAbs, 10(8), 1291-1300. https://doi.org/10.1080/19420862.2018.1511197
Von Kreudenstein, T. S., Escobar-Carbrera, E., Lario, P. I., D'Angelo, I., Brault, K., Kelly, J., . . . Dixit, S. B. (2013). Improving biophysical properties of a bispecific antibody scaffold to aid developability: quality by molecular design. MAbs, 5(5), 646-654. https://doi.org/10.4161/mabs.25632
Winters, C., van Wegen, E. E., Daffertshofer, A., & Kwakkel, G. (2015). Generalizability of the Proportional Recovery Model for the Upper Extremity After an Ischemic Stroke. Neurorehabil Neural Repair, 29(7), 614-622. https://doi.org/10.1177/1545968314562115
Yoo, D., Provchy, J., Park, C., Schulz, C., & Walker, K. (2014). Automated high-throughput protein purification using an AKTApurifier and a CETAC autosampler. J Chromatogr A, 1344, 23-30. https://doi.org/10.1016/j.chroma.2014.04.014

What is claimed is:

1. A method for expressing a multi-chain protein comprising at least two different polypeptide chains comprising
a. providing polynucleotide sequences that encode for the at least two different polypeptides, wherein said polynucleotide sequences are flanked on both 5′ and 3′ sides by a type IIS restriction endonuclease cleavage site followed by a recognition site thereof;
b. providing two or more sets of element sequences; wherein each set of element sequences together comprise at least one functional expression cassette,
wherein each element sequence is flanked on both 5′ and 3′ sides by a type IIS restriction endonuclease cleavage site followed by a recognition site thereof,
wherein the type IIS restriction endonuclease recognition sites and cleavage sites are selected so that the sets of element sequences may be assembled into a functional expression cassette;
c. providing at least one backbone entry vector, wherein the backbone entry vector comprises a plasmid comprising at least one type IIS restriction endonuclease cleavage site followed by a recognition site thereof and sequences for maintenance of the vector in bacterial cells;
d. assembling the polynucleotide sequences that encode for the at least two different polypeptides with the two or more sets of element sequences to generate functional expression cassettes in the backbone entry vector, using a method based on the use of type IIS restriction enzyme digestion and ligation via the cleavage sites and overhangs resulting in ligated vectors comprising at least two functional expression cassettes that are capable of expressing the at least two different polypeptide chains;
e. transfecting mammalian cells with the ligated vectors;
f. comparing the levels of preferred product produced and levels of impurities of each of the at least two polypeptides; and
g. identifying the optimal expression ratio of the two polypeptides for the transfected mammalian cells and identifying the transfected mammalian cells that have the optimal expression ratio of the at least two polypeptide chains.

2. The method according to claim 1, further comprising:
h. identifying the arrangement of vector elements that provides the optimal expression ratio of the at least two polypeptide chains.

3. The method of any preceding claim, wherein the element sequences are selected from the group consisting of control sequence, insulator sequence, bar code DNA sequence, primer sequences. promoter sequences, polyA sequences, and IRES sequences, and a mammalian selectable marker sequence.

4. The method of claim 3, wherein the element sequences comprise at least two promoter sequences and at least two poly A sequences.

5. The method of any preceding claim, wherein the levels of preferred product produced are measured by a method selected from the group consisting of cation exchange chromatography (reduced or non-reduced), mass spectrometry, or a combination thereof.

6. The method of any preceding claim, wherein none of the type IIS restriction endonuclease cleavage sites produce 5′ four nucleotide overbangs selected from the group consisting of GTAA, TCCA, and CACA upon cleavage by the type IIS restriction endonuclease.

7. The method of any preceding claim, wherein the type IIS restriction endonuclease cleavage site is selected from the group consisting of:
AGGT. AGTA, ATCA, CAGT, CCAT, GAAT, GAGG, GGCA, GGTC, TAGC, TCTT, GGAG, and CCAC.

8. The method of any preceding claim, wherein the mammalian cells are selected from the group consisting of CHO cells, CHOK1 cells, DXB-11, DG-44, COS-7, HEK293, BHK, TM4, CV1, VERO-76, HELA, MDCK, BRL 3A, W138, Hep G2, MMT 060562, TRI cells, MRC 5 cells, FS4 cells, and mammalian myeloma cells.

9. The method of any preceding claim, wherein the optimal expression ratio of the at least two polypeptides is selected from the group consisting of 1:1, 1:2, and 1:3.

10. The method of claim 1, wherein the multi-chain protein comprises a first antibody heavy chain, a first antibody light chain, a second antibody heavy chain, and a second antibody light chain, wherein the first antibody heavy chain associates with the first antibody light chain to bind a first antigen or epitope and the second antibody heavy chain associates with the second antibody light chain to bind a second antigen or epitope, wherein the optimal expression ratio of the first antibody heavy chain, the first antibody light chain, the second antibody heavy chain, and the second antibody light chain is 1:1:1:1.

11. The method of claim 1, wherein the multi-chain protein comprises a first antibody heavy chain, a second antibody heavy chain, and a common antibody light chain, wherein the first antibody heavy chain associates with the common antibody light chain to bind a first antigen or epitope and the second antibody heavy chain associates with the common antibody light chain to bind a second antigen or epitope, wherein the optimal expression ratio of the first antibody heavy chain, the second antibody heavy chain, and the common antibody light chain is 1:1:2.

12. The method of claim 1, wherein the multi-chain protein comprises an antibody heavy chain, a first antibody light chain, a modified antibody heavy chain, and a second antibody light chain,
wherein the modified antibody heavy chain comprises, N-terminal to C-terminal, one of the following structures selected from the following group:
a. VH-CH1-binding domain (BD)-hinge-CH2-CH3;
b. BD-VH-CH1-hinge-CH2-CH3; and
c. VH-CH1-hinge-CH2-CH3-BD;
wherein the BD is selected either a single-chain Fv (scFv) or a single-chain Fab (scFab);
wherein the antibody heavy chain associates with the first antibody light chain to bind a first antigen or epitope and the VH of the modified antibody heavy chain associates with the second antibody light chain to bind a second antigen or epitope, wherein the BD binds to a third antigen or epitope, and
wherein the optimal expression ratio of the first antibody heavy chain, the first light chain, the modified antibody heavy chain, and the second antibody light chain is 1:1:1:1.

13. The method of claim 1, wherein the multi-chain protein comprises an antibody heavy chain, a modified antibody heavy chain, and a common antibody light chain,
wherein the modified antibody heavy chain comprises, N-terminal to C-terminal, one of the following structures selected from the following group:
a. VH-CH1-binding domain (BD)-hinge-CH2-CH3;
b. BD-VH-CH1-hinge-CH2-CH3; and
c. VH-CH1-hinge-CH2-CH3-BD;
wherein the BD is selected either a single-chain Fv (scFv) or a single-chain Fab (scFab);
wherein the antibody heavy chain associates with the common antibody light chain to bind a first antigen or epitope and the VH of the modified antibody heavy chain associates with the common antibody light chain to bind the first antigen or epitope, wherein the BD binds to a second antigen or epitope, and
wherein the optimal expression ratio of the first antibody heavy chain, the modified antibody heavy chain, and the common antibody light chain is 1:1:2.

14. A vector comprising the arrangement of vector elements according to claim 2, step h.


SEQUENCE LISTING (12 sequences)

SEQ ID NO: 1 - DNA, 30 bp, synthetic construct
  source 1..30: mol_type = other DNA
cgtctcaccatnnnnnnnnggagactctgc

SEQ ID NO: 2 - DNA, 30 bp, synthetic construct
  source 1..30: mol_type = other DNA
gcagagtctccnnnnnnnnatggtgagacg

SEQ ID NO: 3 - DNA, 11 bp, synthetic construct
  source 1..11: mol_type = other DNA
cgtctcaccat

SEQ ID NO: 4 - DNA, 11 bp, synthetic construct
  source 1..11: mol_type = other DNA
cgtctcaggac

SEQ ID NO: 5 - DNA, 11 bp, synthetic construct
  source 1..11: mol_type = other DNA
gctctcaggtc

SEQ ID NO: 6-7 - skipped (2 entries)

SEQ ID NO: 8 - DNA, 11 bp, synthetic construct
  source 1..11: mol_type = other DNA
gcagagtatgg

SEQ ID NO: 9 - DNA, 11 bp, synthetic construct
  source 1..11: mol_type = other DNA
gcagagtctcc

SEQ ID NO: 10 - DNA, 12 bp, synthetic construct
  source 1..12: mol_type = other DNA
gccagagtgacc

SEQ ID NO: 11-12 - skipped (2 entries)